(19) United States
(12) Patent Application Publication
(10) Pub. No.: US 2021/0095342 A1
(43) Pub. Date: Apr. 1, 2021

(54) FUNCTIONAL TARGETS OF MIR-6891-5P & APPLICATIONS THEREOF

(71) Applicants: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(72) Inventors: Dimitri MONOS, Merion, PA (US); Peter CLARK, Philadelphia, PA (US); Nilesh CHITNIS, Philadelphia, PA (US); Brad JOHNSON, Wynnewood, PA (US); Malek KAMOUN, Wynnewood, PA (US)

(73) Assignees: THE CHILDREN'S HOSPITAL OF PHILADELPHIA, Philadelphia, PA (US); THE TRUSTEES OF THE UNIVERSITY OF PENNSYLVANIA, Philadelphia, PA (US)

(21) Appl. No.: 16/099,243

(22) PCT Filed: May 9, 2017
(86) PCT No.: PCT/US17/31709

§ 371 Date: Nov. 6, 2018

Related U.S. Application Data
(60) Provisional application No. 62/333,633, filed on May 9, 2016.
(60) Provisional application No. 62/428,768, filed on Dec. 1, 2016.

(51) Int. Cl. C12Q 1/6883; C12Q 1/6886; C12N 15/113

(52) U.S. Cl. CPC C12Q 1/6883; C12Q 1/6886; C12N 15/113; C12N 2310/11; C12Q 2600/118; C12N 2310/113; C12Q 2600/178

(57) ABSTRACT

The present disclosure relates to the involvement of HSA-miR-6891-5p in immune and/or inflammatory disorders, as well as the use of agonists/antagonists thereof to treat the same.

This application is a national phase application under 35 U.S.C. § 371 of International Application No. PCT/US2017/031709, filed May 9, 2017, which claims priority to U.S. Provisional Applications, Ser. No. 62/333,633, filed May 9, 2016, and Ser. No. 62/428,768, filed Dec. 1, 2016. The entire contents of each application are hereby incorporated by reference.

Pursuant to 37 C.F.R. 1.821(c), a sequence listing is submitted herewith as an ASCII compliant text file named “CHOPP0010US_ST25.txt”, created on Nov. 5, 2018 and having a size of ˜5 kilobytes. The content of the aforementioned file is hereby incorporated by reference in its entirety.

### BACKGROUND

### 1. Field

The present disclosure relates generally to the fields of medicine, pathology and molecular biology. More particularly, it concerns the role of miRNA function in the development of pathologic disorders. Specifically, the disclosure relates to use of HSA-miR-6891-5p to diagnose or prognose disease, and well as the agonism or antagonism of HSA-miR-6891-5p for treating various disorders.

### 2. Description of Related Art

The major histocompatibility complex (MHC), a 4 Mb region on chromosome 6, encompasses over 180 protein coding genes, including numerous genes involved in innate and adaptive immunity (Horton et al., 2004; Stewart et al., 2004). This region has been shown to harbor the highest number of disease associated genetic variants as compared to any other region of comparable size in the human genome (Clark et al., 2015). Many of these associations lie within the highly polymorphic human leukocyte antigen (HLA) genes (Shiina et al., 2004; Shiina et al., 2009). Given that 90% of causal autoimmune disease variants are located within non-coding regions of the genome (Farh et al., 2015), the non-coding regions of HLA genes may also harbor genomic elements that play a functional role in disease pathogenesis. A search for functional genomic elements within the non-coding regions of HLA genes revealed an annotated microRNA (miRNA), hsa-miR-6891 (miR-6891), which is encoded by intron 4 of HLA-B (Ladewig et al., 2012).

MiRNAs are short (˜22 bp), single stranded, non-coding RNA (ncRNA) transcripts that have been shown to modulate numerous biological processes by regulating the expression of targeted mRNA transcripts through sequence specific miRNA/mRNA interactions, resulting in the degradation or translational suppression of the targeted mRNA transcript (Lodish et al., 2008). Primary miRNA (pri-miRNA) transcripts are generated by RNA polymerase II or III and form precursor miRNA (pre-miRNA) hairpin structures following processing by the Drosha/DGCR8 microprocessor complex (Winter et al., 2009). Alternatively, as is the case with miR-6891, a pre-miRNA hairpin may also be formed independently of the Drosha/DGCR8 microprocessor complex. In these instances, a pre-miRNA is formed from an intronic sequence of a gene following exon splicing of the primary mRNA transcript. Given their biogenesis, such miRNA are termed “mirtrons” and are abundant throughout the genome (Ladewig et al., 2012; Wen et al., 2015). As with other mirtrons, the annotated pre-miRNA hairpin of miR-6891 is believed to be formed from intron 4 of HLA-B following splicing of the primary HLA-B mRNA transcript and is further processed by the Dicer enzyme to produce two mature, single-stranded miRNA transcripts, miR-6891-5p and miR-6891-3p (Ladewig et al., 2012) (FIG. 1). Mature miRNAs bind to mRNA transcripts, forming a heteroduplex that is loaded onto the RNA induced silencing complex (RISC), resulting in post-transcriptional degradation of the targeted mRNA transcript (Jonas and Izaurralde, 2015).

The HLA-B encoded miRNA, miR-6891-5p was initially characterized from a meta-analysis of RNA-seq datasets, with reads from both arms of the hairpin (5′ and 3′ arms together) mapping uniquely to the annotated locus within intron 4 of the HLA-B gene (Ladewig et al., 2012). There is currently no known function of miR-6891-5p.

### SUMMARY

Thus, in accordance with the present disclosure, there is provided a method of identifying a subject having or at risk of developing an immune or inflammatory disorder comprising (a) assessing the level of HSA-miR-6891-5p in a sample from the subject, and (b) comparing the level of HSA-miR-6891-5p in the sample with a normal sample or predetermined control level, wherein an altered level of HSA-miR-6891-5p indicates the existence of or increased risk for an immune or inflammatory disorder. HSA-miR-6891-5p level is elevated or reduced. The sample may be a blood sample.

The inflammatory disorder may be cancer. The immune disorder may be an autoimmune disorder. The immune or inflammatory disorder may be selected from obesity, Crohn's disease, rheumatoid arthritis, asthma, autoimmune thyroid disease, blastic crisis, alopecia areata, multiple sclerosis, autoimmune hepatitis, Addison's disease, type 1 diabetes, type 2 diabetes, bladder cancer, chronic obstructive pulmonary disease, Grave's disease, systemic lupus erythematosus, lung cancer, or Alzheimer's disease. The immune disorder may be IgA nephropathy or IgA deficiency. The subject may be a non-human animal or a human.

In another embodiment, there is provided a method of treating a subject having or at risk of developing an immune or inflammatory disorder comprising administering to the subject an agonist or antagonist of HSA-miR-6891-5p. The method may further comprise (a) assessing the level of HSA-miR-6891-5p in a sample from the subject, and (b) comparing the level of HSA-miR-6891-5p in the sample with a normal sample or predetermined control level. HSA-miR-6891-5p level may be elevated, and an antagonist is administration, or HSA-miR-6891-5p may be reduced, and an agonist is administered.

The inflammatory disorder may be cancer. The immune disorder may be an autoimmune disorder. The immune or inflammatory disorder may be selected from obesity, Crohn's disease, rheumatoid arthritis, asthma, autoimmune thyroid disease, blastic crisis, alopecia areata, multiple sclerosis, autoimmune hepatitis, Addison's disease, type 1 diabetes, type 2 diabetes, bladder cancer, chronic obstructive pulmonary disease, Grave's disease, systemic lupus erythematosus, lung cancer, or Alzheimer's disease. The immune disorder may be IgA nephropathy or IgA deficiency. The subject may be a non-human animal or a human.

The antagonist may be a miR antagomir or antisense molecule. The agonist may be HSA-miR-6891-5p or a mimic thereof. The agonists/antagonist may be formulated in a lipid delivery vehicle. The agonist/antagonist may be a nucleic acid containing at least one non-natural base. The agonist/antagonist may be administered multiple times. The agonist/antagonist may be administered daily, every other day, every third day, every fourth day, every fifth day, every sixth day, weekly or monthly. The agonist/antagonist may be administered continuously over a time period exceeding 24 hours.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the disclosure, and vice versa. Furthermore, compositions and kits of the disclosure can be used to achieve methods of the disclosure.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The disclosure may be better understood by reference to one or more of these drawings in combination with the detailed description of specific embodiments presented herein.

FIG. 1. Predicted biogenesis of HSA-miR-6891. miR-6891 is derived from intron 4 of HLA-B (SEQ ID NO: 1), which upon exon splicing of the HLA-B transcript forms a stable pre-miRNA hairpin structure. The pre-miRNA is then processed by the Dicer enzyme to form two mature miRNA products, HSA-miR-6891-5p (SEQ ID NO: 2) and HSA-miR-6891-3p (SEQ ID NO: 3).

FIGS. 2A-C. HLA-B intron 4 sequence variability and miR-6891 isomiR characterization. (FIG. 2A) There are 384 annotated HLA B alleles with full-length sequence annotation within the IMGT database (release 3.25), with each allele represented by one of eight unique intron 4 sequence motifs. The aligned sequence motifs are provided along with their allele frequency within IMGT and polymorphic positions (highlighted in red). (FIG. 2B) Sequence logo plot depicting the lack of polymorphism within HSA-miR-6891-5p. (FIG. 2C) Sequence logo plot depicting polymorphic sites within HSA-miR-6891-3p at positions 6 and 14 of the mature miRNA.

FIGS. 3A-B. Identification of potential miR-6891-5p targets. COX cells were transduced with lentiviruses expressing either antisense of HSA-miR-6891-5p or scrambled control, and altered mRNA transcript levels were assessed using microarrays. (FIG. 3A) Principal component analysis (PCA) was performed in order to visualize sample clustering and assess the variation among biological replicates (N=3 experimental and 2 controls samples). Clear circles represent the centroid of the sample clusters, and the ellipse represents 2× the standard deviation in the x and y-axis respectively. The first principal component accounts for 76.5% of the variance within the dataset, while the second principal component accounts for 8.6% of the variance within the dataset. (FIG. 3B) Hierarchical clustering of samples based upon identified differentially expressed transcripts from microarray analysis.

FIGS. 4A-E. Validation of miR-6891-5p mediated post-transcriptional regulation of IGHA1 and IGHA2 transcripts. (FIG. 4A) COX cells were transduced with lentiviral constructs expressing either the scrambled control or antisense sequence of miR-6891-5p. Cells were harvested after 48 hours of transduction, total RNA was purified, and both IGHA1 and IGHA2 expression were analyzed by qPCR (ΔΔCt, standard error bars shown, n=3). (FIG. 4B) COX cells (5×10⁸) were transduced with lentiviral constructs expressing either the scrambled control or antisense sequence of miR-6891-5p. After 120 hours, media was collected and analyzed by ELISA using IgA antibody (standard error bars shown, n=3). (FIG. 4C) Predicted binding site and heteroduplex formed between the wild-type (WT) 3′UTR of IGHA2 and miR-6891-5p. The heteroduplex formed with IGHA1 is identical to that shown. (FIG. 4D) Predicted binding site and heteroduplex formed between the mutated (Mut) 3′UTR sequence of IGHA2 and miR-6891-5p. (FIG. 4E) Either the wild-type (WT) or mutated (Mut) 3′ UTR sequence of IGHA2 was cloned downstream of the luciferase reporter, creating two separate constructs. The wild-type or mutant luciferase constructs alone or together with either the miR-6891-5p expression construct (miR overexpression) or the antisense miR-6891-5p expression construct (miR inhibition) were transfected into HEK293T cells. Luciferase assay was performed 24 hours after transfection (standard error bars shown, n=3). All p-values shown are calculated using a t-test.

FIGS. 5A-C. Exploring the role of miR-6891-5p in selective IgA deficiency. (FIG. 5A) Pedigree of affected (proband, black shadowing) and unaffected (white shadowing) family members presented in panels B and C. (FIG. 5B) HLA-B, miR-6891-5p IGHA1 and IGHA2 expression (qPCR) amongst IgA deficient B-LCLs collected from affected individuals and unaffected family members (standard error bars shown, n=3). (FIG. 5C) Selective IgA deficient cell line ID18 was transduced with a lentiviral construct expressing either the antisense miR-6891-5p (miR-6891-5p inhibition) or the scrambled sequence of antisense miR-6891-5p (control). Total RNA was purified and IGHA1 and IGHA2 mRNA transcript levels were analyzed by qPCR (y-axis shown on left of plot, standard error bars shown, n=3). After 24 hours, media was collected and analyzed by ELISA using anti-IgA antibody (y-axis shown on right of plot, standard error bars shown, n=3). All p-values shown are calculated using a t-test.

FIG. 6. Expression of HSA-miR-6891-5p in cultured COX, PGF and HEK293T cells and primary human B-cells purified from total blood. Q-PCR was performed using HSA-miR-6891-5p specific primers and normalized with β-actin Q-PCR data.

FIG. 7. Expression of control (scrambled) and antisense of miR-6891-5p in transduced COX cells. Total RNA was purified and, to confirm the antisense production, the level of mCherry reporter mRNA was analyzed as an indicator of antisense expression. Standard deviation bars show the results of 3 biological replicate experiments. No signal could be detected in untransduced COX cells.

FIG. 8. COX cells were transduced with lentiviral constructs expressing either the scrambled control or antisense sequences of miR-6891-5p. Total RNA was purified and miR-6891-5p expression levels were analyzed by Q-PCR in order to demonstrate that miR-6891-5p expression is comparable between the two conditions and unaffected by transduction. Standard deviation shows results of 3 biological replicate experiments.

### DETAILED DESCRIPTION OF ILLUSTRATIVE EMBODIMENTS

miRNAs are now recognized as significant regulatory elements in eurkayotic gene expression. In their current work, the inventors study the physiological role of miR-6891-5p within B lymphocytes through miR-6891-5p inhibition and transcriptome wide mRNA profiling to identify affected transcripts. Their results indicate that 6891-5p regulates the expression of numerous transcripts including Immunoglobulin Heavy Chain Alpha 1 and 2 (IGHA1 and IGHA2), which was found to be amongst the most enriched mRNA targets of miR-6891-5p. A binding site of miR-6891-5p that is conserved on the 3′UTR of both IGHA1 and IGHA2 was identified by molecular modeling of the two transcripts (IGHA1 and IGHA2 have identical 3′ UTR sequences), and experimentally validated using a luciferase reporter assay. Additional expression profiling of miR-6891-5p and both IGHA1 and IGHA2 transcripts within a cohort of B-LCLs obtained from patients with selective IgA deficiency and unaffected family members reveals a significant increase in miR-6891-5p expression and an attenuation of IGHA1 and IGHA2 expression amongst affected individuals. Furthermore, inhibition of miR-6891-5p within B-LCLs originating from an IgA deficient patient resulted in significantly increased expression of IGHA1 and IGHA2 mRNA and a significant increase in the amount of secreted IgA. These findings indicate a novel physiological role of the HLA-B gene that extends beyond the antigen specific immune responses for which it is well known and raises the possibility that the HLA-B encoded miRNA, miR-6891-5p plays an important role in controlling the expression of many immunologically relevant transcripts. IGHA2 and other HSA-miR-6891-5p targets described herein may prove useful as diagnostic targets, and may also be modulated in disease states by agonists/antagonists of HSA-miR-6891-5p. These and other aspects of the disclosure are discussed in detail below.

### I. miRNAs

A. Background

In 2001, several groups used a novel cloning method to isolate and identify a large group of “microRNAs” (miRNAs) from C. elegans, Drosophila, and humans (Lagos-Quintana et al., 2001; Lau et al., 2001; Lee and Ambros, 2001). Several hundreds of miRNAs have been identified in plants and animals—including humans—which do not appear to have endogenous siRNAs. Thus, while similar to siRNAs, miRNAs are nonetheless distinct.

miRNAs thus far observed have been approximately 21-22 nucleotides in length and they arise from longer precursors, which are transcribed from non-protein-encoding genes. See review of Carrington et al. (2003). The precursors form structures that fold back on each other in self-complementary regions; they are then processed by the nuclease Dicer in animals or DCL1 in plants. miRNA molecules interrupt translation through precise or imprecise base-pairing with their targets.

miRNAs are primarily transcribed by RNA polymerase II and can be derived from individual miRNA genes, from introns of protein coding genes, or from poly-cistronic transcripts that often encode multiple, closely related miRNAs. Pre-miRNAs, generally several thousand bases long are processed in the nucleus by the RNase Drosha into 70- to 100-nt hairpin-shaped precursors. Following transport to the cytoplasm, the hairpin is further processed by Dicer to produce a double-stranded miRNA. The mature miRNA strand is then incorporated into the RNA-induced silencing complex (RISC), where it associates with its target mRNAs by base-pair complementarity to form a heteroduplex of the two single stranded RNA transcripts. In the relatively rare cases in which a miRNA base pairs perfectly with an mRNA target, it promotes mRNA degradation. More commonly, miRNAs form imperfect heteroduplexes with target mRNAs, affecting either mRNA stability or inhibiting mRNA translation.

The 5′ portion of a miRNA spanning bases 2-8, termed the ‘seed’ region, is especially important for target recognition (Krenz and Robbins, 2004; Kiriazis and Krania, 2000). The sequence of the seed, together with phylogenetic conservation of the target sequence, forms the basis for many current target prediction models. Although increasingly sophisticated computational approaches to predict miRNAs and their targets are becoming available, target prediction remains a major challenge and requires experimental validation. Ascribing the functions of miRNAs to the regulation of specific mRNA targets is further complicated by the ability of individual miRNAs to base pair with hundreds of potential high and low affinity mRNA targets and by the targeting of multiple miRNAs to individual mRNAs.

The first miRNAs were identified as regulators of developmental timing in C. elegans, suggesting that miRNAs, in general, might play decisive regulatory roles in transitions between different developmental states by switching off specific targets (Fatkin et al., 2000; Lowes et al., 1997). However, subsequent studies suggest that miRNAs, rather than functioning as on-off “switches,” more commonly function to modulate or fine-tune cell phenotypes by repressing expression of proteins that are inappropriate for a particular cell type, or by adjusting protein dosage. miRNAs have also been proposed to provide robustness to cellular phenotypes by eliminating extreme fluctuations in gene expression.

Research on microRNAs is increasing as scientists are beginning to appreciate the broad role that these molecules play in the regulation of eukaryotic gene expression. The two best understood miRNAs, lin-4 and let-7, regulate developmental timing in C. elegans by regulating the translation of a family of key mRNAs (reviewed in Pasquinelli and Ruvkum, 2002). Numerous miRNAs have been identified in C. elegans, Drosophila, Mus musculus and Homo sapiens. As would be expected for molecules that regulate gene expression, miRNA expression levels have been shown to vary between tissue types, developmental state and disease phenotype. In addition, one study shows a strong correlation between reduced expression of two miRNAs and chronic lymphocytic leukemia, providing a possible link between miRNAs and cancer (Calin et al., 2002). Although the field is still young, there is speculation that miRNAs could be as important as transcription factors in regulating gene expression in higher eukaryotes.

There are a few examples of miRNAs that play critical roles in cell differentiation, early development, and cellular processes like apoptosis and fat metabolism. lin-4 and let-7 both regulate passage from one larval state to another during C. elegans development (Ambros, 2003). mir-14 and bantam are drosophila miRNAs that regulate cell death, apparently by regulating the expression of genes involved in apoptosis (Brennecke et al., 2003, Xu et al., 2003). miR-14 has also been implicated in fat metabolism (Xu et al., 2003). Lsy-6 and miR-273 are C. elegans miRNAs that regulate asymmetry in chemosensory neurons (Chang et al., 2004). Another animal miRNA that regulates cell differentiation is miR-181, which guides hematopoietic cell differentiation (Chen et al., 2004). These molecules represent the full range of animal miRNAs with known functions. Enhanced understanding of the functions of miRNAs will undoubtedly reveal regulatory networks that contribute to normal development, differentiation, inter- and intracellular communication, cell cycle, angiogenesis, apoptosis, and many other cellular processes. Given their important roles in many biological functions, it is likely that miRNAs will offer important points for therapeutic intervention or diagnostic analysis.

Characterizing the functions of biomolecules like miRNAs often involves introducing the molecules into cells or removing the molecules from cells and measuring the result. If introducing a miRNA into cells results in apoptosis, then the miRNA undoubtedly participates in an apoptotic pathway. Methods for introducing and removing miRNAs from cells have been described. Two recent publications describe antisense molecules that can be used to inhibit the activity of specific miRNAs (Meister et al., 2004; Hutvagner et al., 2004), and others have proven their functionality in the heart, where they efficiently knocked-down miR-133 and miR-1 (Care et al. 2007; Yang et al. 2007). Another publication describes the use of plasmids that are transcribed by endogenous RNA polymerases and yield specific miRNAs when transfected into cells (Zeng et al., 2002). These two reagent sets have been used to evaluate single miRNAs.

B. HSA-miR-6891-5p

HSA-miR-6891-5p is derived from intron 4 of the ubiquitously expressed HLA-B transcript following exon splicing. The mature transcript sequence for HSA-miR-6891-5p is uaaggagggggaugagggg (SEQ ID NO: 2).

C. Agonists and Antagonists of miRs

Agonists of HSA-miR-6891-5p will generally take one of three forms. First, there is HSA-miR-6891-5p itself. Such molecules may be delivered to target cells, for example, by injection or infusion, optionally in a delivery vehicle such as a lipid, such as a liposome or lipid emulsion. Second, one may use expression vectors that drive or alter the expression of HSA-miR-6891-5p. The composition and construction of various expression vectors is described elsewhere in the document. Third, one may use agents distinct from HSA-miR-6891-5p that act to up-regulate, stabilize or otherwise enhance the activity of HSA-miR-6891-5p, including small molecules. Such molecules include “mimetics,” molecules which mimic the function, and possibly form of HSA-miR-6891-5p, but are distinct in chemical structure.

Antagonism of miRNA function may, in example, be achieved by “antagomirs.” Initially described by Krützfeldt and colleagues (Krützfeldt et al., 2005), antagomirs are single-stranded, chemically-modified ribonucleotides that are at least partially complementary to the miRNA sequence. Antagomirs may comprise one or more modified nucleotides, such as 2′-O-methyl-sugar modifications. In some embodiments, antagomirs comprise only modified nucleotides. Antagomirs may also comprise one or more phosphorothioate linkages resulting in a partial or full phosphorothioate backbone. To facilitate in vivo delivery and stability, the antagomir may be linked to a cholesterol moiety at its 3′ end. Antagomirs suitable for inhibiting miRNAs may be about 14 to about 50 nucleotides in length, about 14 to about 30 nucleotides in length, and 14 to about 25 nucleotides in length. “Partially complementary” refers to a sequence that is at least about 75%, 80%, 85%, 90%, 95%, 96%, 97%, 98%, or 99% complementary to a target polynucleotide sequence. The antagomirs may be at least about 75%, 80%, 85%, 90%, 95%, 96%, 97%, 98%, or 99% complementary to a mature miRNA sequence. In some embodiments, the antagomir may be substantially complementary to a mature miRNA sequence, that is at least about 95%, 96%, 97%, 98%, or 99% complementary to a target polynucleotide sequence. In other embodiments, the antagomirs are 100% complementary to the mature miRNA sequence.

Inhibition of miRNA function may also be achieved by administering antisense oligonucleotides. The antisense oligonucleotides may be ribonucleotides or deoxyribonucleotides. Preferably, the antisense oligonucleotides have at least one chemical modification. Antisense oligonucleotides may be comprised of one or more “locked nucleic acids.” “Locked nucleic acids” (LNAs) are modified ribonucleotides that contain an extra bridge between the 2′ and 4′ carbons of the ribose sugar moiety resulting in a “locked” conformation that confers enhanced thermal stability to oligonucleotides containing the LNAs. Alternatively, the antisense oligonucleotides may comprise peptide nucleic acids (PNAs), which contain a peptide-based backbone rather than a sugar-phosphate backbone. Other chemical modifications that the antisense oligonucleotides may contain include, but are not limited to, sugar modifications, such as 2′-O-alkyl (e.g., 2′-O-methyl, 2′-O-methoxyethyl), 2′-fluoro, and 4′ thio modifications, and backbone modifications, such as one or more phosphorothioate, morpholino, or phosphonocarboxylate linkages (see, for example, U.S. Pat. Nos. 6,693,187 and 7,067,641, which are herein incorporated by reference in their entireties). In some embodiments, suitable antisense oligonucleotides are 2′-O-methoxyethyl “gapmers” which contain 2′-O-methoxyethyl-modified ribonucleotides on both 5′ and 3′ ends with at least ten deoxyribonucleotides in the center. These “gapmers” are capable of triggering RNase H-dependent degradation mechanisms of RNA targets. Other modifications of antisense oligonucleotides to enhance stability and improve efficacy, such as those described in U.S. Pat. No. 6,838,283, which is herein incorporated by reference in its entirety, are known in the art and are suitable for use in the methods of the disclosure. Particular antisense oligonucleotides useful for inhibiting the activity of microRNAs are about 19 to about 25 nucleotides in length. Antisense oligonucleotides may comprise a sequence that is at least partially complementary to a mature miRNA sequence, e.g., at least about 75%, 80%, 85%, 90%, 95%, 96%, 97%, 98%, or 99% complementary to a mature miRNA sequence. In some embodiments, the antisense oligonucleotide may be substantially complementary to a mature miRNA sequence, that is at least about 95%, 96%, 97%, 98%, or 99% complementary to a target polynucleotide sequence. In one embodiment, the antisense oligonucleotide comprises a sequence that is 100% complementary to a mature miRNA sequence.

Another approach for inhibiting the function of a miRNA is administering an inhibitory RNA molecule having at least partial sequence identity to the mature miR sequence. The inhibitory RNA molecule may be a double-stranded, small interfering RNA (siRNA) or a short hairpin RNA molecule (shRNA) comprising a stem-loop structure. The double-stranded regions of the inhibitory RNA molecule may comprise a sequence that is at least partially identical, e.g., about 75%, 80%, 85%, 90%, 95%, 96%, 97%, 98%, or 99% identical, to the mature miRNA sequence. In some embodiments, the double-stranded regions of the inhibitory RNA comprise a sequence that is at least substantially identical to the mature miRNA sequence. “Substantially identical” refers to a sequence that is at least about 95%, 96%, 97%, 98%, or 99% identical to a target polynucleotide sequence. In other embodiments, the double-stranded regions of the inhibitory RNA molecule may contain 100% identity to the target miRNA sequence.

In other embodiments of the disclosure, inhibitors of a miRNA may be inhibitory RNA molecules, such as ribozymes, siRNAs, or shRNAs. In one embodiment, an inhibitor of HSA-miR-6891-5p is an inhibitory RNA molecule comprising a double-stranded region, wherein the double-stranded region comprises a sequence having 100% identity to the mature miR sequence. In some embodiments, inhibitors are inhibitory RNA molecules which comprise a double-stranded region, wherein said double-stranded region comprises a sequence of at least about 75%, 80%, 85%, 90%, 95%, 96%, 97%, 98%, or 99% identity to the mature miR sequence.

### II. IGHA2

IgA (immunoglobulin alpha) is the major immunoglobulin class in bodily secretions. It can serve both to defend against local infection, and to prevent access of foreign antigens to the general immunologic system. It has been demonstrated to play a role in the antibacterial humoral response, the B cell receptor signaling pathway, the complement activation (classical pathway), the Fc-epsilon receptor signaling pathway, the Fc-gamma receptor signaling pathway involved in phagocytosis, glomerular filtration, phagocytosis, positive regulation of respiratory burst and retina homeostasis.

IgA is present in normal human serum at about 20% of the amount of IgG. It is, however, the most abundant Ig in secretions, and as such, it is the most extensively produced Ig in humans. It is exists in two isotopic forms—IgA1 and IgA2. Both of these antibodies exists in monomeric and di-/polymeric configurations, largely depending on where they are produced in the body. Most IgA is produced by mucosal lymphocytes and J-chain associated dimers. Polymeric IgA (e.g., tetrameric) also contains a highly glycosylated protein called secretory factor (SC) that is complexed with IgA during the

IgA2 differs from IgA1 in only 22 amino acids, mostly due to a deletion in IgA2 of 13 residues from the hinge region. The absence of this region makes IgA2 resistant to a number of bacterial proteinases that cleave IgA2. IgA2 variants include IgA2m(1) and IgA2m(2), and tthse differ. IgA2m(1) lacks the disulphide bond between the light and heavy chain, thereby allowing two light chains to be linked to each other. Under denaturing conditions, the molecule spints tino heavy chain and light chain dimers.

### III. METHODS OF TREATMENT

A. Pharmacological Therapeutic Agents and Administration

The present disclosure addresses therapies, e.g., treatment of various conditions. In various embodiments, the inhibitory agents of the present disclosure are formulated for administration in pharmacologically acceptable vehicles, such as parenteral, topical, aerosal, liposomal, nasal or ophthalmic preparations. In certain embodiments, formulations may be designed for oral or topical administration. It is further envisioned that formulations of nucleic acids encoding cytoskeletal stabilizing proteins and any other agents that might be delivered may be formulated and administered in a manner that does not require that they be in a single pharmaceutically acceptable carrier. In those situations, it would be clear to one of ordinary skill in the art the types of diluents that would be proper for the proposed use of the polypeptides and any secondary agents required.

The phrases “pharmaceutically” or “pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the compositions, vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue or surface is available via that route. This includes oral, nasal, or topical. Alternatively, administration may be by introcular, intra-hepatic, orthotopic, intradermal, subcutaneous, intramuscular, intraperitoneal or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

B. IgA2 Related Disease States

In particular aspects, the present disclosure provides for the diagnosis and treatment of diseases that involve the dysregulation of IgA2. Two particular disease states—IgA nephropathy and IgA deficiency—are discussed below.

1. IgA Nephropathy

IgA nephropathy (IgAN), also known as IgA nephritis, Berger disease (and variations), or synpharyngitic glomerulonephritis, is a disease of the kidney (or nephropathy), specifically it is a form of glomerulonephritis or an inflammation of the glomeruli of the kidney.

IgA nephropathy is the most common glomerulonephritis worldwide. Primary IgA nephropathy is characterized by deposition of the IgA antibody in the glomerulus. There are other diseases associated with glomerular IgA deposits, the most common being Henoch-Schonlein purpura (HSP), which is considered by many to be a systemic form of IgA nephropathy. HSP presents with a characteristic purpuric skin rash, arthritis, and abdominal pain and occurs more commonly in young adults (16-35 yrs old). HSP is associated with a more benign prognosis than IgA nephropathy. In IgA nephropathy there is a slow progression to chronic kidney failure in 25-30% of cases during a period of 20 years.

Men are affected three times as often as women. There is also a striking geographic variation in the prevalence of IgA nephropathy throughout the world. It is the most common glomerular disease in the Far East and Southeast Asia, comprising almost half of all the patients with glomerular disease. However, it comprises only about 25% of the proportion in European and about 10% among North Americans, with African-Americans having a very low prevalence of about 2%. A confounding factor in this analysis is the existing policy of screening and use of kidney biopsy as an investigative tool. School children in Japan undergo routine urinalysis (as do Army recruits in Singapore) and any suspicious abnormality is pursued with a kidney biopsy, which might partly explain the high observed incidence of IgA nephropathy in those countries.

The classic presentation (in 40-50% of the cases) is episodic hematuria which usually starts within a day or two of a non-specific upper respiratory tract infection (hence synpharyngitic) as opposed to post-streptococcal glomerulonephritis which occurs some time (weeks) after initial infection. Less commonly gastrointestinal or urinary infection can be the inciting agent. All of these infections have in common the activation of mucosal defenses and hence IgA antibody production. Groin pain can also occur. The gross hematuria resolves after a few days, though microscopic hematuria may persist. These episodes occur on an irregular basis every few months and in most patients eventually subsides (although it can take many years). Renal function usually remains normal, though rarely, acute kidney failure may occur (see below). This presentation is more common in younger adults.

A smaller proportion (20-30%), usually the older population, have microscopic hematuria and proteinuria (less than 2 gram/day). These patients may not have any symptoms and are only clinically found if a doctor decides to take a urine sample. Hence, the disease is more commonly diagnosed in situations where screening of urine is compulsory, e.g., schoolchildren in Japan.

Very rarely (5% each), the presenting history is:
Nephrotic syndrome (3-3.5 grams of protein loss in the urine, associated with a poorer prognosis)
Acute kidney failure (either as a complication of the frank hematuria, when it usually recovers, or due to rapidly progressive glomerulonephritis which often leads to chronic kidney failure)
Chronic kidney failure (no previous symptoms, presents with anemia, hypertension and other symptoms of kidney failure, in people who probably had longstanding undetected microscopic hematuria and/or proteinuria)

A variety of systemic diseases are associated with IgA nephropathy such as liver failure, celiac disease, rheumatoid arthritis, reactive arthritis, ankylosing spondylitis and HIV. Diagnosis of IgA nephropathy and a search for any associated disease occasionally reveals such an underlying serious systemic disease. Occasionally, there are simultaneous symptoms of Henoch-Schonlein purpura; see below for more details on the association. Some HLA alleles have been suspected along with complement phenotypes as being genetic factors.

For an adult patient with isolated hematuria, tests such as ultrasound of the kidney and cystoscopy are usually done first to pinpoint the source of the bleeding. These tests would rule out kidney stones and bladder cancer, two other common urological causes of hematuria. In children and younger adults, the history and association with respiratory infection can raise the suspicion of IgA nephropathy. A kidney biopsy is necessary to confirm the diagnosis. The biopsy specimen shows proliferation of the mesangium, with IgA deposits on immunofluorescence and electron microscopy. However, patients with isolated microscopic hematuria (i.e., without associated proteinuria and with normal kidney function) are not usually biopsied since this is associated with an excellent prognosis. A urinalysis will show red blood cells, usually as red cell urinary casts. Proteinuria, usually less than 2 grams per day, also may be present. Other renal causes of isolated hematuria include thin basement membrane disease and Alport syndrome, the latter being a hereditary disease associated with hearing impairment and eye problems.

Other blood tests done to aid in the diagnosis include CRP or ESR, complement levels, ANA, and LDH. Protein electrophoresis and immunoglobulin levels can show increased IgA in 50% of all patients.

Histologically, IgA nephropathy may show mesangial widening and focal and segmental inflammation. Diffuse mesangial proliferation or crescentic glomerulonephritis may also be present. Immunoflourescence shows mesangial deposition of IgA often with C3 and properdin and smaller amounts of other immunoglobulins (IgG or IgM). Early components of the classical complement pathway (C1 q or C4) are usually not seen. Electron microscopy confirms electron-dense deposits in the mesangium that may extend to the subendothelial area of adjacent capillary walls in a small subset of cases, usually those with focal proliferation.

The disease derives its name from deposits of Immunoglobulin A (IgA) in a granular pattern in the mesangium (by immunofluorescence), a region of the renal glomerulus. The mesangium by light microscopy may be hypercellular and show increased deposition of extracellular matrix proteins.

There is no clear known explanation for the accumulation of the IgA. Exogenous antigens for IgA have not been identified in the kidney, but it is possible that this antigen has been cleared before the disease manifests itself. It has also been proposed that IgA itself may be the antigen.

A recently advanced theory focuses on abnormalities of the IgA1 molecule. IgA1 is one of the two immunoglobulin subclasses (the other is IgD) that is O-glycosylated on a number of serine and threonine residues in a special proline-rich hinge region. Aberrant glycosylation of IgA appears to lead to polymerisation of the IgA molecules in tissues, especially the glomerular mesangium. A similar mechanism has been claimed to underlie Henoch-Schonlein purpura (HSP), a vasculitis that mainly affects children and can feature renal involvement that is almost indistinguishable from IgA nephritis. However, human studies have found that degalactosylation of IgA1 occurs in patients with IgA nephropathy in response only to gut antigen exposures (not systemic), and occurs in healthy people to a lesser extent. This strongly suggests degalactosylation of IgA1 is a result of an underlying phenomenon (abnormal mucosal antigen handling) and not the ultimate cause of IgA nephropathy. Prevailing evidence suggests that both galactose-deficient o-glycans in the hinge region of IgA1 and synthesis and binding of antibodies against IgA1 are required for immunoglobulin complexes to form and accumulate in glomeruli.

From the fact that IgAN can recur after renal transplant it can be postulated that the disease is caused by a problem in the immune system rather than the kidney itself. Remarkably, the IgA1 that accumulates in the kidney does not appear to originate from the mucosa-associated lymphoid tissue (MALT), which is the site of most upper respiratory tract infections, but from the bone marrow. This, too, suggests an immune pathology rather than direct interference by outside agents.

Since IgA nephropathy commonly presents without symptoms through abnormal findings on urinalysis, there is considerable possibility for variation in any population studied depending upon the screening policy. Similarly, the local policy for performing kidney biopsy assumes a critical role; if it is a policy to simply observe patients with isolated bloody urine, a group with a generally favourable prognosis will be excluded. If, in contrast, all such patients are biopsied, then the group with isolated microscopic hematuria and isolated mesangial IgA will be included and ‘improve’ the prognosis of that particular series.

Nevertheless, IgA nephropathy, which was initially thought to be a benign disease, has been shown to have not-so-benign long term outcomes. Though most reports describe IgA nephropathy as having an indolent evolution towards either healing or renal damage, a more aggressive course is occasionally seen associated with extensive crescents, and presenting as acute kidney failure. In general, the entry into chronic kidney failure is slow as compared to most other glomerulonephritides—occurring over a time scale of 30 years or more (in contrast to the 5 to 15 years in other glomerulonephritides). This may reflect the earlier diagnosis made due to frank hematuria.

Complete remission, i.e., a normal urinalysis, occurs rarely in adults, in about 5% of cases. Thus, even in those with normal renal function after a decade or two, urinary abnormalities persist in the great majority. In contrast, 30-50% of children may have a normal urinalysis at the end of 10 years. However, given the very slow evolution of this disease, the longer term (20-30 years) outcome of such patients is not yet established. Overall, though the renal survival is 80-90% after 10 years, at least 25% and maybe up to 45% of adult patients will eventually develop end stage renal disease.

The ideal treatment for IgAN would remove IgA from the glomerulus and prevent further IgA deposition. This goal still remains a remote prospect. There are a few additional caveats that have to be considered while treating IgA nephropathy. IgA nephropathy has a very variable course, ranging from a benign recurrent hematuria up to a rapid progression to chronic kidney failure. Hence the decision on which patients to treat should be based on the prognostic factors and the risk of progression. Also, IgA nephropathy recurs in transplants despite the use of ciclosporin, azathioprine or mycophenolate mofetil and steroids in these patients. There are persisting uncertainties, due to the limited number of patients included in the few controlled randomized studies performed to date, which hardly produce statistically significant evidence regarding the heterogeneity of IgA nephropathy patients, the diversity of study treatment protocols, and the length of follow-up.

Patients with isolated hematuria, proteinuria<1 g/day and normal renal function have a benign course and are generally just followed up annually. In cases where tonsillitis is the precipitating factor for episodic hematuria, tonsillectomy has been claimed to reduce the frequency of those episodes. However, it does not reduce the incidence of progressive kidney failure. Also, the natural history of the disease is such that episodes of frank hematuria reduce over time, independent of any specific treatment. Similarly, prophylactic antibiotics have not been proven to be beneficial. Dietary gluten restriction, used to reduce mucosal antigen challenge, also has not been shown to preserve kidney function. Phenytoin has also been tried without any benefit.

A subset of IgA nephropathy patients, who have minimal change disease on light microscopy and clinically have nephrotic syndrome, show an exquisite response to steroids, behaving more or less like minimal change disease. In other patients, the evidence for steroids is not compelling. Short courses of high dose steroids have been proven to lack benefit. However, in patients with preserved renal function and proteinuria (1-3.5 g/day), a recent prospective study has shown that 6 months regimen of steroids may lessen proteinuria and preserve renal function. However, the risks of long-term steroid use have to be weighed in such cases. It should be noted that the study had 10 years of patient follow-up data, and did show a benefit for steroid therapy; there was a lower chance of reaching end-stage renal disease (renal function so poor that dialysis was required) in the steroid group. Importantly, angiotensin-converting enzyme inhibitors were used in both groups equally.

Cyclophosphamide had been used in combination with anti-platelet/anticoagulants in unselected IgA nephropathy patients with conflicting results. Also, the side effect profile of this drug, including long term risk of malignancy and sterility, made it an unfavorable choice for use in young adults. However, one recent study, in a carefully selected high risk population of patients with declining GFR, showed that a combination of steroids and cyclophosphamide for the initial 3 months followed by azathioprine for a minimum of 2 years resulted in a significant preservation of renal function. Other agents such as mycophenolate mofetil, cyclosporin and mizoribine have also been tried with varying results.

A study from Mayo Clinic did show that long term treatment with omega-3 fatty acids results in reduction of progression to kidney failure, without, however, reducing proteinuria in a subset of patients with high risk of worsening kidney function. However, these results have not been reproduced by other study groups and in two subsequent meta-analyses. However, fish oil therapy does not have the drawbacks of immunosuppressive therapy. Also, apart from its unpleasant taste and abdominal discomfort, it is relatively safe to consume.

The events that tend to progressive kidney failure are not unique to IgA nephropathy and non-specific measures to reduce the same would be equally useful. These include low-protein diet and optimal control of blood pressure. The choice of the antihypertensive agent is open as long as the blood pressure is controlled to desired level. However, Angiotensin converting enzyme inhibitors and Angiotensin II receptor antagonists are favoured due to their anti-proteinuric effect.

Though various associations have been described, no consistent pattern pointing to a single susceptible gene has been yet identified. Associations described include those with C4 null allele, factor B Bf alleles, MHC antigens and IgA isotypes. ACE gene polymorphism (D allele) is associated with progression of kidney failure, similar to its association with other causes of chronic kidney failure. However, more than 90% of cases of IgA nephropathy are sporadic, with a few large pedigrees described from Kentucky and Italy.

Male gender, proteinuria (especially >2 g/day), hypertension, smoking, hyperlipidemia, older age, familial disease and elevated creatinine concentrations are markers of a poor outcome. Frank hematuria has shown discordant results with most studies showing a better prognosis, perhaps related to the early diagnosis, except for one group which reported a poorer prognosis. Proteinuria and hypertension are the most powerful prognostic factors in this group.

There are certain other features on kidney biopsy such as interstitial scarring which are associated with a poor prognosis. ACE gene polymorphism has been recently shown to have an impact with the DD genotype associated more commonly with progression to kidney failure.

2. IgA Deficiency

Selective immunoglobulin A (IgA) deficiency (SIgAD) is a genetic immunodeficiency. People with this deficiency lack immunoglobulin A (IgA), a type of antibody that protects against infections of the mucous membranes lining the mouth, airways, and digestive tract. It is defined as an undetectable serum IgA level in the presence of normal serum levels of IgG and IgM. It is the most common of the primary antibody deficiencies.

Prevalence varies by population, but is on the order of up to 1 in 333 people, making it relatively common for a genetic disease. It is more common in males than in females.

In IgA-deficient patients, the common finding is a maturation defect in B cells to produce IgA. In IgA deficiency, B cells express IgA; however, they are of immature phenotype with the coexpression of IgM and IgD, and they cannot fully develop into IgA-secreting plasma cells. There is an inherited inability to produce immunoglobulin A (IgA), a part of the body's defenses against infection at the body's surfaces (mainly the surfaces of the respiratory and digestive systems). As a result, bacteria at these locations are somewhat more able to cause disease.

About 85-90% of IgA-deficient individuals are asymptomatic, although the reason for lack of symptoms is relatively unknown and continues to be a topic of interest and controversy. Some patients with IgA deficiency have a tendency to develop recurrent sinopulmonary infections, gastrointestinal infections and disorders, allergies, autoimmune conditions, and malignancies. These infections are generally mild and would not usually lead to an in-depth workup except when unusually frequent. They may present with severe reactions including anaphylaxis to blood transfusions or intravenous immunoglobulin due to the presence of IgA in these blood products. When suspected, the diagnosis can be confirmed by laboratory measurement of IgA level in the blood. Patients have an increased susceptibility to pneumonia and recurrent episodes of other respiratory infections and a higher risk of developing autoimmune diseases in middle age.

Although it has some similarities to common variable immunodeficiency, it does not present the same lymphocyte subpopulation abnormalities. It may anyway progress to CVID. Those patients with selective immunoglobulin A deficiency may be prone to recurrent infections when on hemodialysis.

The treatment consists of identification of comorbid conditions, preventive measures to reduce the risk of infection, and prompt and effective treatment of infections. Infections in an IgA-deficient person are treated as usual (i.e., with antibiotics). There is no treatment for the underlying disorder.

There is a historical popularity in using intravenous immunoglobulin (IVIG) to treat SIGAD, but the consensus is that there is no evidence that IVIG treats this condition. In cases where a patient presents SIGAD and another condition which is treatable with IVIG, then a physician may treat the other condition with IVIG. The use of IVIG to treat SIGAD without first demonstrating an impairment of specific antibody formation is extremely controversial.

Prognosis is excellent, although there is an association with autoimmune disease. Of note, selective IgA deficiency can complicate the diagnosis of one such condition, celiac disease, as the deficiency masks the high levels of certain IgA antibodies usually seen in celiac disease. Selective IgA deficiency occurs in 1 of 39 to 57 patients with celiac disease. This is much higher than the prevalence of selective IgA deficiency in the general population, which is estimated to be approximately 1 in 400 to 18 500, depending on ethnic background. The prevalence of celiac disease in patients with selective IgA deficiency ranges from 10% to 30%, depending on the evaluated population.

As opposed to the related condition CVID, selective IgA deficiency is not associated with an increased risk of cancer.

C. Combined Therapy

In another embodiment, it is envisioned to use the agonists/antagonists of the present disclosure in combination with other therapeutic modalities. Thus, in addition to the therapies described above, one may also provide to the patient more “standard” pharmaceutical therapies. Combinations may be achieved by contacting cells, tissues or subjects with a single composition or pharmacological formulation that includes both agents, or by contacting the cell with two distinct compositions or formulations, at the same time, wherein one composition includes the agonist/antagonist and the other includes the other agent. Alternatively, the therapy using an agonist/antagonist may precede or follow administration of the other agent(s) by intervals ranging from minutes to weeks. In embodiments where the other agent and agonist/antagonist are applied separately to the cell, one would generally ensure that a significant period of time did not expire between each delivery, such that the agent and the agonist/antagonist would still be able to exert an advantageously combined effect on the cell, tissue or subject. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a modulator of miR, or the other agent will be desired. In this regard, various combinations may be employed. By way of illustration, where the agonist/antagonist(s) is “A” and the other agent is “B,” the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated.

Particularly useful combination therapies will include anti-cancer, anti-inflammatory and immunomodulatory therapies.

### IV. DETECTION METHODS

One embodiment of the present disclosure comprises a method for detecting variation in the expression of HSA-miR-6891-5p, or in the structure of the HSA-miR-6891-5p coding sequence. Also contemplated are epigenetic modifications, such as methylation of promoter regions that control HSA-miR-6891-5p expression. Such assays may comprise determining that level of HSA-miR-6891-5p in a sample, or determining specific alterations in the expressed product. The biological sample can be any tissue or fluid that can contain cells. Various embodiments include cells of the skin, muscle, facia, brain, prostate, breast, endometrium, lung, head & neck, pancreas, small intestine, blood cells, liver, testes, ovaries, colon, skin, stomach, esophagus, spleen, lymph node, bone marrow or kidney. Other embodiments include fluid samples such as peripheral blood, lymph fluid, ascites, serous fluid, pleural effusion, nipple aspirates, sputum, cerebrospinal fluid, lacrimal fluid, stool or urine.

Nucleic acid used is isolated from cells contained in the biological sample, according to standard methodologies. The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals.

Following detection, one may compare the results seen in a given patient with a statistically significant reference group of normal patients and patients that have HSA-miR-6891-5p-related pathologies. In this way, it is possible to correlate the amount or structure of HSA-miR-6891-5p detected with various clinical states. “Alterations” should be read as including deletions, insertions, point mutations and duplications. Point mutations result in stop codons, frameshift mutations or amino acid substitutions. Somatic mutations are those occurring in non-germline tissues. Germ-line tissue can occur in any tissue and are inherited. Mutations or epigenetic modifications in and outside the coding region also may affect the amount of HSA-miR-6891-5p produced, both by altering the transcription of the gene or in destabilizing or otherwise altering the processing of either the transcript (mRNA) or protein. It is contemplated that other mutations in the HSA-miR-6891-5p coding sequence may be identified in accordance with the present disclosure. A variety of different assays are contemplated in this regard, including but not limited to, fluorescent in situ hybridization (FISH), direct DNA sequencing, PFGE analysis, Southern or Northern blotting, single-stranded conformation analysis (SSCA), RNAse protection assay, allele-specific oligonucleotide (ASO), dot blot analysis, denaturing gradient gel electrophoresis, RFLP and PCR™-SSCP. Some specific examples are provided below.

A. SNP Analysis

The methods described herein include determining the identity, e.g., the specific nucleotide, presence or absence, of a SNP. The SNPs may be a gain of function mutation, a loss of function mutation, or have no effect. It is within the skill of those in the field to ascertain whether a mutation adds, detracts or has no change on the activity of a molecule examined. Samples that are suitable for use in the methods described herein contain genetic material, e.g., genomic DNA (gDNA). Genomic DNA is typically extracted from biological samples. The sample itself will typically include a tumor biopsy removed from the subject. Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

In some cases, a biological sample may be processed for DNA isolation. For example, DNA in a cell or tissue sample can be separated from other components of the sample. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. The sample can be concentrated and/or purified to isolate DNA. All samples obtained from a subject, including those subjected to any sort of further processing, are considered to be obtained from the subject. Routine methods can be used to extract genomic DNA from a biological sample, including, for example, phenol extraction. Alternatively, genomic DNA can be extracted with kits such as the QIAamp® Tissue Kit (Qiagen, Chatsworth, Calif.) and the Wizard® Genomic DNA purification kit (Promega). Non-limiting examples of sources of samples include urine, blood, and tissue.

The presence or absence of the SNP can be determined using methods known in the art. For example, gel electrophoresis, capillary electrophoresis, size exclusion chromatography, sequencing, and/or arrays can be used to detect the presence or absence of specific response alleles. Amplification of nucleic acids, where desirable, can be accomplished using methods known in the art, e.g., PCR. In one example, a sample (e.g., a sample comprising genomic DNA), is obtained from a subject. The DNA in the sample is then examined to determine the identity of an allele as described herein, i.e., by determining the identity of one or more alleles associated with a selected response. The identity of an allele can be determined by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

Other methods of nucleic acid analysis can include direct manual sequencing (U.S. Pat. No. 5,288,644); automated fluorescent sequencing; single-stranded conformation polymorphism assays (SSCP); clamped denaturing gel electrophoresis (CDGE); two-dimensional gel electrophoresis (2DGE or TDGE); conformational sensitive gel electrophoresis (CSGE); denaturing gradient gel electrophoresis (DGGE); denaturing high performance liquid chromatography (DHPLC); infrared matrix-assisted laser desorption/ionization (IR-MALDI) mass spectrometry (WO 99/57318); mobility shift analysis; restriction enzyme analysis; quantitative real-time PCR; heteroduplex analysis; chemical mismatch cleavage (CMC); RNase protection assays; use of polypeptides that recognize nucleotide mismatches, e.g., E. coli mutS protein; allele-specific PCR, and combinations of such methods. See, e.g., U.S. Patent Publication No. 2004/0014095, which is incorporated herein by reference in its entirety.

Sequence analysis can also be used to detect specific polymorphic variants. For example, polymorphic variants can be detected by sequencing exons, introns, 5′ untranslated sequences, or 3′ untranslated sequences. A sample comprising DNA or RNA is obtained from the subject. PCR or other appropriate methods can be used to amplify a portion encompassing the polymorphic site, if desired. The sequence is then ascertained, using any standard method, and the presence of a polymorphic variant is determined. Real-time pyrophosphate DNA sequencing is yet another approach to detection of polymorphisms and polymorphic variants. Additional methods include, for example, PCR amplification in combination with denaturing high performance liquid chromatography (dHPLC).

PCR refers to procedures in which target nucleic acid (e.g., genomic DNA) is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. Other amplification methods that may be employed include the ligase chain reaction (LCR), transcription amplification, self-sustained sequence replication, and nucleic acid based sequence amplification (NASBA). Guidelines for selecting primers for PCR amplification are well known in the art.

In some cases, PCR conditions and primers can be developed that amplify a product only when the variant allele is present or only when the wild type allele is present (MSPCR or allele-specific PCR). For example, patient DNA and a control can be amplified separately using either a wild-type primer or a primer specific for the variant allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products would be detected in each reaction.

In some embodiments, a peptide nucleic acid (PNA) probe can be used instead of a nucleic acid probe in the hybridization methods described above. PNA is a DNA mimetic with a peptide-like, inorganic backbone, e.g., N-(2-aminoethyl)glycine units, with an organic base (A, G, C, T or U) attached to the glycine nitrogen via a methylene carbonyl linker. The PNA probe can be designed to specifically hybridize to a nucleic acid comprising a polymorphic variant.

In some cases, allele-specific oligonucleotides can also be used to detect the presence of a polymorphic variant. For example, polymorphic variants can be detected by performing allele-specific hybridization or allele-specific restriction digests. Allele specific hybridization is an example of a method that can be used to detect sequence variants, including complete genotypes of a subject (e.g., a mammal such as a human). An “allele-specific oligonucleotide” (also referred to herein as an “allele-specific oligonucleotide probe”) is an oligonucleotide that is specific for particular a polymorphism can be prepared using standard methods. Allele-specific oligonucleotide probes typically can be approximately 10-50 base pairs, preferably approximately 15-30 base pairs, that specifically hybridize to a nucleic acid region that contains a polymorphism. Hybridization conditions are selected such that a nucleic acid probe can specifically bind to the sequence of interest, e.g., the variant nucleic acid sequence. Such hybridizations typically are performed under high stringency as some sequence variants include only a single nucleotide difference. In some cases, dot-blot hybridization of amplified oligonucleotides with allele-specific oligonucleotide (ASO) probes can be performed.

In some embodiments, allele-specific restriction digest analysis can be used to detect the existence of a polymorphic variant of a polymorphism, if alternate polymorphic variants of the polymorphism result in the creation or elimination of a restriction site. Allele-specific restriction digests can be performed in the following manner. A sample containing genomic DNA is obtained from the individual and genomic DNA is isolated for analysis. For nucleotide sequence variants that introduce a restriction site, restriction digest with the particular restriction enzyme can differentiate the alleles. In some cases, polymerase chain reaction (PCR) can be used to amplify a region comprising the polymorphic site, and restriction fragment length polymorphism analysis is conducted. The digestion pattern of the relevant DNA fragment indicates the presence or absence of a particular polymorphic variant of the polymorphism and is therefore indicative of the subject's response allele. For sequence variants that do not alter a common restriction site, mutagenic primers can be designed that introduce a restriction site when the variant allele is present or when the wild type allele is present. For example, a portion of a nucleic acid can be amplified using the mutagenic primer and a wild-type primer, followed by digest with the appropriate restriction endonuclease.

In some embodiments, fluorescence polarization template-directed dye-terminator incorporation (FP-TDI) is used to determine which of multiple polymorphic variants of a polymorphism is present in a subject. Rather than involving use of allele-specific probes or primers, this method employs primers that terminate adjacent to a polymorphic site, so that extension of the primer by a single nucleotide results in incorporation of a nucleotide complementary to the polymorphic variant at the polymorphic site.

In some cases, DNA containing an amplified portion may be dot-blotted, using standard methods, and the blot contacted with the oligonucleotide probe. The presence of specific hybridization of the probe to the DNA is then detected. Specific hybridization of an allele-specific oligonucleotide probe (specific for a polymorphic variant indicative of a predicted response to a method of treating an SSD) to DNA from the subject is indicative of a subject's response allele.

Methods of nucleic acid analysis to detect polymorphisms and/or polymorphic variants can include, e.g., microarray analysis. Hybridization methods, such as Southern analysis, Northern analysis, or in situ hybridizations, can also be used (see, Ausubel et al., 2003). To detect microdeletions, fluorescence in situ hybridization (FISH) using DNA probes that are directed to a putatively deleted region in a chromosome can be used. For example, probes that detect all or a part of a microsatellite marker can be used to detect microdeletions in the region that contains that marker.

In some embodiments, it is desirable to employ methods that can detect the presence of multiple polymorphisms (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to the methods provided herein.

Nucleic acid probes can be used to detect and/or quantify the presence of a particular target nucleic acid sequence within a sample of nucleic acid sequences, e.g., as hybridization probes, or to amplify a particular target sequence within a sample, e.g., as a primer. Probes have a complimentary nucleic acid sequence that selectively hybridizes to the target nucleic acid sequence. In order for a probe to hybridize to a target sequence, the hybridization probe must have sufficient identity with the target sequence, i.e., at least 70% (e.g., 80%, 90%, 95%, 98% or more) identity to the target sequence. The probe sequence must also be sufficiently long so that the probe exhibits selectivity for the target sequence over non-target sequences. For example, the probe will be at least 20 (e.g., 25, 30, 35, 50, 100, 200, 300, 400, 500, 600, 700, 800, 900 or more) nucleotides in length. In some embodiments, the probes are not more than 30, 50, 100, 200, 300, 500, 750, or 1000 nucleotides in length. Probes are typically about 20 to about 1×10⁶ nucleotides in length. Probes include primers, which generally refers to a single-stranded oligonucleotide probe that can act as a point of initiation of template-directed DNA synthesis using methods such as PCR (polymerase chain reaction), LCR (ligase chain reaction), etc., for amplification of a target sequence.

The probe can be a test probe such as a probe that can be used to detect polymorphisms in a region described herein (e.g., an allele associated with treatment response as described herein). In some embodiments, the probe can bind to another marker sequence associated with SZ, SPD, or SD as described herein or known in the art.

Control probes can also be used. For example, a probe that binds a less variable sequence, e.g., repetitive DNA associated with a centromere of a chromosome, can be used as a control. Probes that hybridize with various centromeric DNA and locus-specific DNA are available commercially, for example, from Vysis, Inc. (Downers Grove, Ill.), Molecular Probes, Inc. (Eugene, Oreg.), or from Cytocell (Oxfordshire, UK). Probe sets are available commercially such from Applied Biosystems, e.g., the Assays-on-Demand SNP kits. Alternatively, probes can be synthesized, e.g., chemically or in vitro, or made from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via the polymerase chain reaction (PCR). See, for example, U.S. Pat. No. 5,491,224.

In some embodiments, the probes are labeled, e.g., by direct labeling, with a fluorophore, an organic molecule that fluoresces after absorbing light of lower wavelength/higher energy. A directly labeled fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, e.g., U.S. Pat. No. 5,491,224.

Fluorophores of different colors can be chosen such that each probe in a set can be distinctly visualized. For example, a combination of the following fluorophores can be used: 7-amino-4-methylcoumarin-3-acetic acid (AMCA), TEXAS RED™ (Molecular Probes, Inc., Eugene, Oreg.), 5-(and -6)-carboxy-X-rhodamine, lissamine rhodamine B, 5-(and -6)-carboxyfluorescein, fluorescein-5-isothiocyanate (FITC), 7-di ethyl aminocoumarin-3-carboxylic acid, tetramethylrhodamine-5-(and -6)-isothiocyanate, 5-(and -6)-carboxytetramethylrhodamine, 7-hydroxycoumarin-3-carboxylic acid, 6-[fluorescein 5-(and -6)-carboxamidolhexanoic acid, N-(4,4-difluoro-5,7-dimethyl-4-bora-3a,4a diaza-3-indacenepropionic acid, eosin-5-isothiocyanate, erythrosin-5-isothiocyanate, and CASCADE™ blue acetylazide (Molecular Probes, Inc., Eugene, Oreg.). Fluorescently labeled probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the probes. Fluorescence-based arrays are also known in the art.

In other embodiments, the probes can be indirectly labeled with, e.g., biotin or digoxygenin, or labeled with radioactive isotopes such as ³²P and ³H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

B. Comparative Genomic Hybridization

Comparative genomic hybridization is a molecular cytogenetic method for analysing copy number variations (CNVs) relative to ploidy level in the DNA of a test sample compared to a reference sample, without the need for culturing cells. The aim of this technique is to quickly and efficiently compare two genomic DNA samples arising from two sources, which are most often closely related, because it is suspected that they contain differences in terms of either gains or losses of either whole chromosomes or subchromosomal regions (a portion of a whole chromosome). This technique was originally developed for the evaluation of the differences between the chromosomal complements of solid tumor and normal tissue, and has an improved resolution of 5-10 megabases compared to the more traditional cytogenetic analysis techniques of Giemsa banding and fluorescence in situ hybridization (FISH) which are limited by the resolution of the microscope utilized.

This is achieved through the use of competitive fluorescence in situ hybridization. In short, this involves the isolation of DNA from the two sources to be compared, most commonly a test and reference source, independent labelling of each DNA sample with a different fluorophores (fluorescent molecules) of different colors (usually red and green), denaturation of the DNA so that it is single stranded, and the hybridization of the two resultant samples in a 1:1 ratio to a normal metaphase spread of chromosomes, to which the labelled DNA samples will bind at their locus of origin. Using a fluorescence microscope and computer software, the differentially colored fluorescent signals are then compared along the length of each chromosome for identification of chromosomal differences between the two sources. A higher intensity of the test sample color in a specific region of a chromosome indicates the gain of material of that region in the corresponding source sample, while a higher intensity of the reference sample colour indicates the loss of material in the test sample in that specific region. A neutral color (yellow when the fluorophore labels are red and green) indicates no difference between the two samples in that location.

CGH is only able to detect unbalanced chromosomal abnormalities. This is because balanced chromosomal abnormalities, such as reciprocal translocations, inversions or ring chromosomes, do not affect copy number that is detected by CGH technologies. CGH does, however, allow for the exploration of all 46 human chromosomes in single test and the discovery of deletions and duplications, even on the microscopic scale which may lead to the identification of candidate genes to be further explored by other cytological techniques.

Through the use of DNA microarrays in conjunction with CGH techniques, the more specific form of array CGH (aCGH) has been developed, allowing for a locus-by-locus measure of CNV with increased resolution as low as 100 kilobases. This improved technique allows for the etiology of known and unknown conditions to be discovered.

In making assessments of decreased or increased copy number, one will reference the copy number of genes that do not vary in copy number, such as housekeeping genes including β-actin and GAPDH.

C. Northern Blot

The northern blot is a technique used in molecular biology research to study gene expression by detection of RNA (or isolated mRNA) in a sample. With northern blotting it is possible to observe cellular control over structure and function by determining the particular gene expression levels during differentiation, morphogenesis, as well as abnormal or diseased conditions. Northern blotting involves the use of electrophoresis to separate RNA samples by size and detection with a hybridization probe complementary to part of or the entire target sequence. The term ‘northern blot’ actually refers specifically to the capillary transfer of RNA from the electrophoresis gel to the blotting membrane. However, the entire process is commonly referred to as northern blotting.

A general blotting procedure starts with extraction of total RNA from a homogenized tissue sample or from cells. Eukaryotic mRNA can then be isolated through the use of oligo (dT) cellulose chromatography to isolate only those RNAs with a poly(A) tail. RNA samples are then separated by gel electrophoresis. Since the gels are fragile and the probes are unable to enter the matrix, the RNA samples, now separated by size, are transferred to a nylon membrane through a capillary or vacuum blotting system.

A nylon membrane with a positive charge is the most effective for use in northern blotting since the negatively charged nucleic acids have a high affinity for them. The transfer buffer used for the blotting usually contains formamide because it lowers the annealing temperature of the probe-RNA interaction, thus eliminating the need for high temperatures, which could cause RNA degradation. Once the RNA has been transferred to the membrane, it is immobilized through covalent linkage to the membrane by UV light or heat. After a probe has been labeled, it is hybridized to the RNA on the membrane. Experimental conditions that can affect the efficiency and specificity of hybridization include ionic strength, viscosity, duplex length, mismatched base pairs, and base composition. The membrane is washed to ensure that the probe has bound specifically and to prevent background signals from arising. The hybrid signals are then detected by X-ray film and can be quantified by densitometry. To create controls for comparison in a northern blot samples not displaying the gene product of interest can be used after determination by microarrays or RT-PCR.

The RNA samples are most commonly separated on agarose gels containing formaldehyde as a denaturing agent for the RNA to limit secondary structure. The gels can be stained with ethidium bromide (EtBr) and viewed under UV light to observe the quality and quantity of RNA before blotting. Polyacrylamide gel electrophoeresis with urea can also be used in RNA separation but it is most commonly used for fragmented RNA or microRNAs. An RNA ladder is often run alongside the samples on an electrophoresis gel to observe the size of fragments obtained but in total RNA samples the ribosomal subunits can act as size markers. Since the large ribosomal subunit is 28S (approximately 5 kb) and the small ribosomal subunit is 18S (approximately 2 kB) two prominent bands appear on the gel, the larger at close to twice the intensity of the smaller.

Probes for northern blotting are composed of nucleic acids with a complementary sequence to all or part of the RNA of interest, they can be DNA, RNA, or oligonucleotides with a minimum of 25 complementary bases to the target sequence. RNA probes (riboprobes) that are transcribed in vitro are able to withstand more rigorous washing steps preventing some of the background noise. Commonly cDNA is created with labelled primers for the RNA sequence of interest to act as the probe in the northern blot. The probes must be labelled either with radioactive isotopes (³²P) or with chemiluminescence in which alkaline phosphatase or horseradish peroxidase break down chemiluminescent substrates producing a detectable emission of light. The chemiluminescent labelling can occur in two ways: either the probe is attached to the enzyme, or the probe is labelled with a ligand (e.g., biotin) for which the antibody (e.g., avidin or streptavidin) is attached to the enzyme. X-ray film can detect both the radioactive and chemiluminescent signals and many researchers prefer the chemiluminescent signals because they are faster, more sensitive, and reduce the health hazards that go along with radioactive labels. The same membrane can be probed up to five times without a significant loss of the target RNA.

D. Fluorescence In Situ Hybridization and Chromogenic In Situ Hybridization

Fluorescence in situ hybridization (FISH) can be used for molecular studies. FISH is used to detect highly specific DNA probes which have been hybridized to chromosomes using fluorescence microscopy. The DNA probe is labeled with fluorescent or non fluorescent molecules which are then detected by fluorescent antibodies. The probes bind to a specific region or regions on the target chromosome. The chromosomes are then stained using a contrasting color, and the cells are viewed using a fluorescence microscope.

Each FISH probe is specific to one region of a chromosome, and is labeled with fluorescent molecules throughout its length. Each microscope slide contains many metaphases. Each metaphase consists of the complete set of chromosomes, one small segment of which each probe will seek out and bind itself to. The metaphase spread is useful to visualize specific chromosomes and the exact region to which the probe binds. The first step is to break apart (denature) the double strands of DNA in both the probe DNA and the chromosome DNA so they can bind to each other. This is done by heating the DNA in a solution of formamide at a high temperature (70-75° C.). Next, the probe is placed on the slide and the slide is placed in a 37° C. incubator overnight for the probe to hybridize with the target chromosome. Overnight, the probe DNA seeks out its target sequence on the specific chromosome and binds to it. The strands then slowly reanneal. The slide is washed in a salt/detergent solution to remove any of the probe that did not bind to chromosomes and differently colored fluorescent dye is added to the slide to stain all of the chromosomes so that they may then be viewed using a fluorescent light microscope. Two, or more different probes labeled with different fluorescent tags can be mixed and used at the same time. The chromosomes are then stained with a third color for contrast. This gives a metaphase or interphase cell with three or more colors which can be used to detect different chromosomes at the same time, or to provide a control probe in case one of the other target sequences are deleted and a probe cannot bind to the chromosome. This technique allows, for example, the localization of genes and also the direct morphological detection of genetic defects.

The advantage of using FISH probes over microsatellite instability to test for copy is that the:
(a) FISH is easily and rapidly performed on cells of interest and can be used on paraffin-embedded, or fresh or frozen tissue allowing the use of microdissection;
(b) specific gene changes can be analyzed on a cell by cell basis in relationship to centomeric probes so that true homozygosity versus heterozygosity of a DNA sequence can be evaluated (use of PCR™ for microsatellite instability may permit amplification of surrounding normal DNA sequences from contamination by normal cells in a homozygously deleted region imparting a false positive impression that the allele of interest is not deleted);
(c) PCR cannot identify amplification of genes; and
(d) FISH using bacterial artificial chromosomes (BACs) permits easy detection and localization on specific chromosomes of genes of interest which have been isolated using specific primer pairs.

Chromogenic in situ hybridzation (CISH) enables the gain genetic information in the context of tissue morphology using methods already present in histology labs. CISH allows detection of gene amplification, chromosome translocations and chromosome number using conventional enzymatic reactions under the brightfield microscope on formalin-fixed, paraffin-embedded (FFPE) tissues. U.S. Publication No. 2009/0137412, incorporated herein by reference. The scanning may be performed, for example, on an automated scanner with Fluorescence capabilities (Bioview System, Rehovot, Israel).

### V. KITS

Any of the compositions described herein may be comprised in a kit. The kits may be designed for either therapeutic or diagnostic purposes. In a non-limiting example, an individual miRNA agonist/antagonists (e.g., expression construct, antagomir, LNA) is included in a kit. The kit may also include one or more transfection reagent(s) to facilitate delivery of the agonist/antagonist to cells. Alternatively, the kit may contain reagents designed to measure miRNA levels, such as probes and primers, as well as enzymes for performing diagnostic reactions (polymerazes, detectable enzymes and labels, etc.).

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present disclosure also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present disclosure will also typically include a means for containing the vials in close confinement for commercial sale, such as, e.g., injection and/or blow-molded plastic containers into which the desired vials are retained.

Such kits may also include components that preserve or maintain the miRNA or that protect against its degradation. Such components may be RNAse-free or protect against RNAses. Such kits generally will comprise, in suitable means, distinct containers for each individual reagent or solution. A kit will also include instructions for employing the kit components as well the use of any other reagent not included in the kit. Instructions may include variations that can be implemented.

It is contemplated that such reagents are embodiments of kits of the disclosure. Such kits, however, are not limited to the particular items identified above and may include any reagent used for the manipulation or characterization of miRNA.

### VI. VECTORS FOR CLONING, GENE TRANSFER AND EXPRESSION

Within certain embodiments expression vectors are employed to express nucleic acid agonist/antagonists, such as miRs, antisense molecules. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression construct” is meant to include any type of genetic construct containing a nucleic acid coding for a gene product in which part or all of the nucleic acid encoding sequence is capable of being transcribed. Generally, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In other embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 1 and 2 list several regulatory elements that may be employed, in the context of the present disclosure, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 1 and Table 2). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 1
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; Imbra |
| | et al., 1986; Kadesch et al., 1986; Wang et al., 1986; |
| | Ondek et al., 1987; Kuhl et al., 1987; Schaffner et |
| | al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky et |
| | al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe et |
| | al., 1988; Rosen et al., 1988; Berkhout et al., 1989; |
| | Laspia et al., 1989; Sharp et al., 1989; Braddock et |
| | al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart et |
| | | al., 1985; Imagawa et al., |
| | | 1987, Karin et al., 1987; |
| | | Angel et al., 1987b; |
| | | McNeall et al., 1989 |
| MMTV (mouse mammary | Glucocorticoids | Huang et al., 1981; Lee et |
| tumor virus) | | al., 1981; Majors et al., |
| | | 1983; Chandler et al., |
| | | 1983; Ponta et al., 1985; |
| | | Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | ElA, SV40 Large T Antigen | Taylor et al., 1989, 1990a, |
| | | 1990b |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

Where a cDNA insert is employed, one will typically desire to include a polyadenylation signal to effect proper polyadenylation of the gene transcript. The nature of the polyadenylation signal is not believed to be crucial to the successful practice of the disclosure, and any such sequence may be employed such as human growth hormone and SV40 polyadenylation signals. Also contemplated as an element of the expression cassette is a terminator. These elements can serve to enhance message levels and to minimize read through from the cassette into other sequences.

B. Selectable Markers

In certain embodiments of the disclosure, the cells contain nucleic acid constructs of the present disclosure, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

C. Delivery of Expression Vectors

There are a number of ways in which expression vectors may be introduced into cells. In certain embodiments of the disclosure, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990). Defective hepatitis B viruses also are useful as expression vectors (Horwich et al., 1990).

Several non-viral methods for the transfer of expression constructs into cultured mammalian cells also are contemplated by the present disclosure. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the disclosure, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the disclosure for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the eye, liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present disclosure.

In a further embodiment of the disclosure, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments of the disclosure, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In a particular example, the oligonucleotide may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

### VII. DEFINITIONS

The term “treatment” or grammatical equivalents encompasses the improvement and/or reversal of the symptoms of disease. “Improvement in the physiologic function” of the eye may be assessed using any of the measurements described herein.

The term “compound” refers to any chemical entity, pharmaceutical, drug, and the like that can be used to treat or prevent a disease, illness, sickness, or disorder of bodily function.

Compounds comprise both known and potential therapeutic compounds. A compound can be determined to be therapeutic by screening using the screening methods of the present disclosure. A “known therapeutic compound” refers to a therapeutic compound that has been shown (e.g., through animal trials or prior experience with administration to humans) to be effective in such treatment.

As used herein, the terms “antagonist” and “inhibitor” refer to molecules, compounds, or nucleic acids that inhibit the action of a factor. Antagonists may or may not be homologous to these natural compounds in respect to conformation, charge or other characteristics.

Antagonists may have allosteric effects that prevent the action of an agonist. Alternatively, antagonists may prevent the function of the agonist. Antagonists and inhibitors may include proteins, nucleic acids, carbohydrates, small molecule pharmaceuticals or any other molecules that bind or interact with a receptor, molecule, and/or pathway of interest.

As used herein, the term “agonist” refers to molecules or compounds that mimic or promote the action of a “native” or “natural” compound. Agonists may be homologous to these natural compounds in respect to conformation, charge or other characteristics. Agonists may include proteins, nucleic acids, carbohydrates, small molecule pharmaceuticals or any other molecules that interact with a molecule, receptor, and/or pathway of interest.

### VIII. EXAMPLES

The following examples are included to further illustrate various aspects of the disclosure. It should be appreciated by those of skill in the art that the techniques disclosed in the examples which follow represent techniques and/or compositions discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials & Methods

HSA-miR-6891 IsomiR Characterization & Sequence Conservation. Full-length annotated HLA-B allele sequences were obtained from IMGT (v 3.23.0) and aligned using Clustal Omega (Sievers et al., 2011). The multiple sequence alignment (MSA) was subsequently used to characterize the sequence variability within intron 4 across HLA-B alleles. Sequence logo plots for regions encoding the two mature miRNAs, HSA-miR-6891-5p and HSA-miR-6891-3p were generated using MATLAB (R2014b) in order to visualize sequence variability within the mature miRNA products. Sequence conservation of the pre-miRNA (HSA-miR-6891) hairpin was determined using BLAST (blastn 2.4.0) against the reference genomic sequence database (refseq genomic) with the following parameter settings: word size of 28, expected value of 10, hitlist size of 100, Match/Mismatch scores of 1/-2, gapcosts of 0, low complexity filter on, filter string set to L;m; and genetic code set to 1.

Cell Culture. COX cells (Traherne et al., 2006) were obtained from the International Histocompatibility Working Group, Seattle, Wash. (IHW09022) (world-wide web at ihwg.org/hla/index.html). PGF cells (Traherne et al., 2006) were obtained from the Coriell Biorepository (Cat #GM03107). Cells were cultured in RPMI-1640 medium with 15% FBS (Sigma Cat #F2442-500ML). HEK 293T cells (a gift from Xianxin Hua in the Department of Cancer Biology at the University of Pennsylvania, Perelman School of Medicine) were cultured in DMEM (Cat #10-013-CV) media with 10% FBS. Primary B-cells were purified from peripheral blood using EasySep™ Direct Human B Cell Isolation Kit from Stemcells Technologies Cat #19674 and directly used for RNA purification. Selective IgA deficiency patient cell lines (ID 000018, ID000036, ID000038, ID000057) and cell lines from unaffected, related family members (ID000037 and ID000058) were originally collected and characterized as part of an initiative by the US Immunodeficiency Network (USIDNET) and purchased from the Coriell Biorepository.

HSA-miR-6891-5p Inhibition. An “inhibition” lentivirus was generated in cultured HEK 293T cells by transfecting with a pEZX-am03 vector (Genecoepia) containing an HSA-miR-6891-5p antisense insert under the control of a CMV promoter. A lentivirus containing similar insert but with a “scrambled” sequence (i.e., random sequence changes all the bases in the seed region) was similarly generated in HEK 293T cells. Media was discarded after 24 hours post-transfection and packaging media was added to the plate. Scrambled and HSA-miR-6891-5p knock-down viruses were collected every 24 hours for 2 days.

For transduction, 1.5×10⁵ COX cells were plated in 6 well plates, and 2 ml of fresh scrambled or miR-6891-5p knock-down lentivirus was added along with 4 mg/ml polybrene. The plate was centrifuged at 2,500 rpm for 90 minutes. After 10 hours, 2 ml of additional virus with polybrene was added and the plate was centrifuged at 2,500 rpm for 90 minutes. After 16 hours, 2 ml of media was discarded and 2 ml of fresh virus and polybrene were added, and the plate was centrifuged at 2,500 rpm for 90 minutes. Transduction was allowed to continue for an additional 24 hours before cells were collected for RNA extraction. RNA was purified using the miRNeasy kit (Qiagen).

Microarray Analysis. Total RNA extracted from each of the biological replicates of transfected COX cells for both conditions (i.e., inhibition and scrambled control) was used to generate sense-strand cDNA using the Ambion® WT Expression Kit for Affymetrix® GeneChip® Whole Transcript (WT) Expression Arrays (P/N 4425209). From each of these reactions, 5.5 ug of sense-strand cDNA was fragmented and labeled using the Affymetrix GeneChip WT Terminal Labeling and Hybridization Kit (PN 702880). Fragmented and labeled sense-strand cDNA (3.25 μg) was hybridized to an Affymetrix Human Gene 2.0ST Array. Arrays were washed on an Affymetrix GeneChip Fluidics Station 450 using fluidics protocol FS450_0002 and scanned on Affymetrix GeneChip Scanner 3000.

Raw data (CEL) files were imported and processed within MATLAB (R2014b). Raw data was first background adjusted using the robust multi-array average (RMA) procedure, followed by quantile normalization with median polishing and probe level summarization using a custom CDF annotation file (Bolstad et al., 2003; Irizarry et al., 2003a; Irizarry et al., 2003b; Dai et al., 2005; Sandberg and Larsson, 2007). Those probes on the array with null values for at least one sample were removed so as not to confound subsequent analysis. Principal component analysis (PCA) of the normalized dataset was performed within MATLAB (R2014b) in order to visualize sample clustering and identify sample outliers (Shieh and Hung, 2009). Differentially expressed transcripts were identified between the miR-6891-5p inhibition samples and control samples (scrambled vector) using significant analysis of microarrays (SAM) (Tusher et al., 2001). Differentially expressed transcripts were identified using a false discovery rate (FDR) cutoff of 0.05 (Storey, 2002; Storey and Tibshirani, 2003) and a fold-change cutoff of 2. Hierarchical clustering of differentially expressed transcripts for each replicate was performed using MATLAB (R2014b). Functional enrichment was performed using DAVID (Huang da et al., 2009). Significant gene ontology (GO) biological processes (BP_FAT) and molecular functions (MF FAT) were determined using a p-value cutoff of 0.05. Raw microarray data is publicly available and may be accessed via NCBI GEO (link provided upon acceptance).

Computational Prediction of HSA-miR-6891-5p Targets. Computationally predicted mRNA targets of HSA-miR-6891-5p were identified throughout the entirety of every annotated gene using miRWalk2.0 with default parameters and every available database, including miRWalk, miRDB, PITA, MicroT4, miRMap, RNA22, miRanda, miRNAMap, RNAhybrid, miRBridge, PICTAR2 and Targetscan (Dweep and Gretz, 2015). The set of genes with a computationally predicted miRNA binding site for miR-6891-5p were then intersected with the set of targets identified by microarray analysis.

HLA Genotyping. Genomic DNA was extracted from the IgA deficient B-LCLs using the Qiagen Gentra Puregene Blood Kit (Cat No./ID: 158389). Sequencing libraries were generated for each sample using the Omixon Holotype HLA Genotyping Kit as previously described (Duke et al., 2016). The library was then denatured with NaOH and diluted to a final concentration of 8 pM for optimal cluster density and 600 μl was loaded into the MiSeq reagent cartridge (v2 500 cycle kit). Samples were de-multiplexed on the instrument and the resulting FASTQ files were used for further analysis. All samples were genotyped at the HLA-B locus using Omixon Target (version 1.8). High resolution HLA-B genotyping results may be found in Table S4.

Quantitative PCR. Total RNA was extracted from cells using a Qiagen miRNeasy kit (Cat #217084) per manufacturer's protocol (Gordanpour et al., 2012). Total RNA was reverse transcribed using the Qiagen miRNA Reverse Transcription kit (Cat #218160). qPCR was performed on cDNA generated by reverse transcription using a miSCRIPT SYBR Green PCR kit (Cat #21803). Primers for HSA-miR-6891-5p were obtained from Qiagen (Cat #MS00048202). Primers for IGHA1, IGHA2, β-actin and HLA-B (sequences provided below) were obtained from IDT. Selective IgA patient cells were HLA genotyped and qPCR primers were designed to amplify all genotyped HLA-B mRNA transcripts. Selective IgA deficiency cells were harvested and total RNA was purified using Qiagen miRneasy kit. This RNA was used for qPCR using HLA-B and miR-6891-5p primers. Data was normalized to actin. Significance was assessed using an unpaired one tailed t-test. Primer sequences are:

| | |
|---|---|
| i) | IGHA1 |
| | (SEQ ID NO: 21) |
| | a. Forward 5′-TTCCCTCAACTCCACCTACC-3′ |
| | (SEQ ID NO: 22) |
| | b. Reverse 5′-CGTGAGGTTCGCTTCTGAAC-3′ |
| ii) | IGHA2 |
| | (SEQ ID NO: 23) |
| | a. Forward 5′-GAGACCTTCACCTGCACTG-3′ |
| | (SEQ ID NO: 24) |
| | b. Reverse 5′-TGTGTTTCCGGATTTTGTGATGT-244 3′ |
| iii) | β-actin |
| | (SEQ ID NO: 25) |
| | a. Forward 5′-AGAGCTACGAGCTGCCTGAC-3′ |
| | (SEQ ID NO: 26) |
| | b. Reverse 5′-AGCACTGTGTTGGCGTACAG-3′ |
| iv) | HSA-miR-6891-5p mCherry Reporter |
| | (SEQ ID NO: 27) |
| | a. Forward 5′-CAGACCGCCAAGCTGAA-3′ |
| | (SEQ ID NO: 28) |
| | b. Reverse 5′-GAGCCGTACATGAACTGAGG-3′ |
| v) | HLA-B mRNA |
| | (SEQ ID NO: 29) |
| | a. Forward 5′-GTCCTAGCAGTTGTGGTCATC-3′ |
| | (SEQ ID NO: 30) |
| | b. Reverse 5′-CAAGCTGTGAGAGACACATCAGA-3′ |

IgA ELISA. COX and PGF cells were cultured in RPMI-1640 media. After 72 and/or 120 hours, media was collected and IgA secretion was analyzed using Ready-SET Go ELISA kit (Wu et al., 2014; Sebastian et al., 2016) (Cat #88-50600) from Affymetrix (CA) per manufacturer's protocol. Significance was assessed using the one-tailed t-test.

Luciferase Assay. The complete (48 nucleotide) 3′ UTR sequence of the IGHA1 gene (which is identical to the 3′UTR sequence of the IGHA2 gene), containing the HSA-miR-6891-5p binding site, was synthesized with Pme I and Xba I sites on either end (IDT) and gel purified using a QIAquick Gel Extraction Kit (Qiagen Cat #28704). The product was ligated into the pmiRGLO plasmid (Promega, Wis.) digested with Pme I and Xba I (New England Biolabs, MA) downstream of the PGK promoter and luciferase gene.

For the luciferase assay, 1× 106 HEK 293T cells were cultured in multi-well plates and, after 24 hours, were transfected with either the wild-type IGHA1 3′UTR or mutant IGHA1 3′UTR construct using Fugene 6 (Promega Cat #E2691) Some of these cells were also transfected with either HSA-miR-6891-5p antisense or overexpression constructs. After 24 hours, the cells were assayed for luciferase activity using the Dual-Luciferase® Reporter Assay System (Promega, Cat #E1910) (Chitnis et al., 2012). For each measurement, firefly luciferase data was normalized to renilla luciferase. Significance was assessed using Student's t-test.

### Example 2—Results

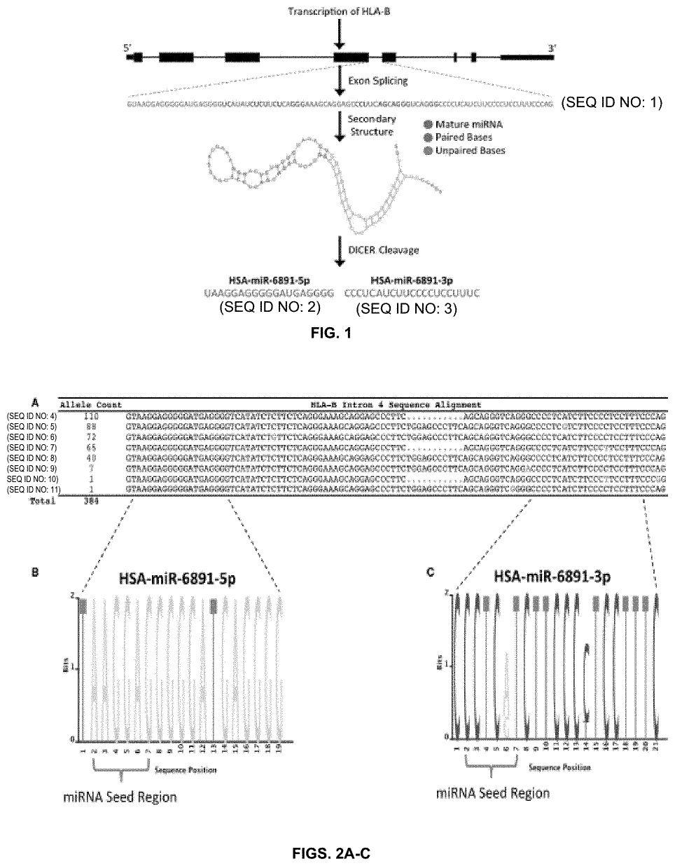

miR-6891 Sequence Variability. Following transcript splicing, intron 4 of HLA-B is predicted to form a pre-miRNA hairpin that is further processed by the Dicer enzyme into two mature miRNA products, miR-6891-5p and miR-6891-3p (Ladewig et al., 2012) (FIG. 1). Given the highly polymorphic nature of the HLA-B locus, the inventors explored miR-6891 sequence variants (isomiRs) by interrogating the sequences of intron 4 among the 384 full-length annotated HLA-B alleles in the international ImMunoGeneTics database (IMGT/HLA, release 3.25) (Robinson et al., 2013). Among those, only eight unique sequence motifs were observed (FIG. 2A). Remarkably and despite the very polymorphic nature of the HLA-B gene, there is no sequence variation within miR-6891-5p (FIG. 2B) and only two polymorphic sites within the mature miR-6891-3p arm, occurring at positions 6 and 14 of the mature miRNA (FIG. 2C). Each of these intronic sequences form stable pre-miRNA hairpin structures with secondary structure minimum free energy values ranging from −43 to −54 kcal/mol. The inventors selected miR-6891-5p for additional study because its conserved sequence suggests an important biological role. The pre-miRNA hairpin sequence of hsa-miR-6891 is evolutionarily conserved, with 90% sequence identity amongst 6 primate species including Homo sapiens, Gorilla gorilla, Nomascus leucogenys, Chlorocebus sabaeus, Macaca nemestrina and Macaca mulatta. In contrast, the closest homolog of hsa-miR-6891 within the mouse genome, which lies within intron 5 of the H2-T10 gene, has only 48% (45/93 base positions identical) sequence conservation with hsa-miR-6891 and there is no annotated miRNA encoded within this locus (miRbase release 21).

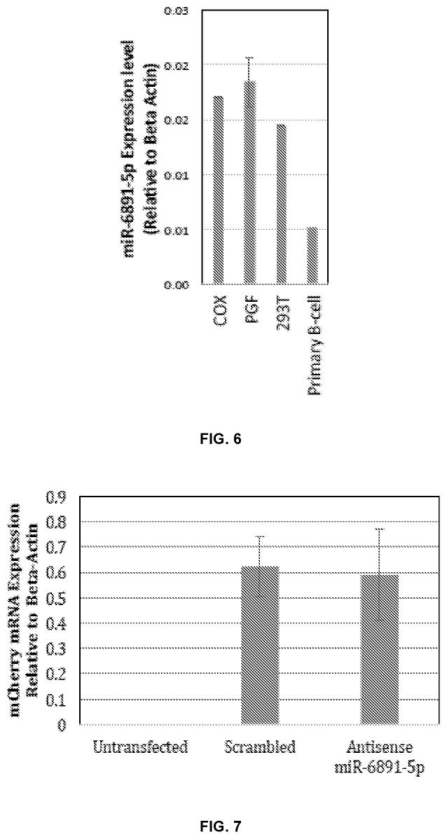

miR-6891-5p Targeting in B-Lymphocytes. To study the function of miR-6891-5p, an appropriate in vitro cell model was first identified by examining the expression level of miR-6891-5p within two B-LCLs, PGF and COX, as well as immortalized HEK293T cells and primary B-lymphocytes (FIG. 6). qPCR results indicate that miR-6891-5p is expressed in every cell type analyzed, with B-LCLs exhibiting the highest and most uniform expression of miR-6891-5p across biological replicates. For this reason, B-LCLs (COX cells) were selected as a model system to further study the role of miR-6891-5p.

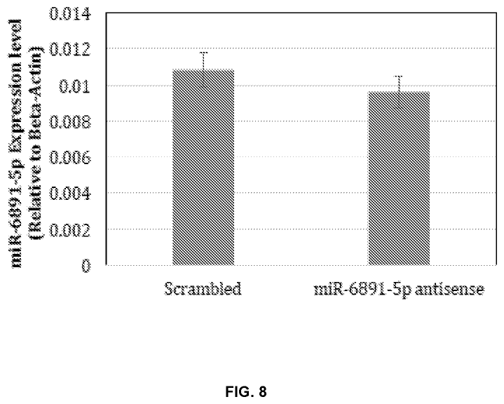

To identify putative target transcripts of miR-6891-5p, the inventors transduced COX cells with a lentiviral construct expressing the anti-sense transcript of miR-6891-5p to inhibit the activity of miR-6891-5p. Upon transduction, transcripts targeted by miR-6891-5p are expected to be more abundant within the transduced cells since miR-dependent degradation has been inhibited. The experimental design included COX cells transduced with either the lentiviral construct expressing the anti-sense sequence (inhibition) or scrambled anti-sense sequence (control) of miR-6891-5p. Adequate and comparable expression of lentiviral constructs from both experimental conditions was observed (FIGS. 7-8). Affymetrix Human Gene 2.0ST Arrays were used to assess transcript expression levels between the miR-6891-5p inhibition and control sample groups. Principal component analysis (PCA; FIG. 3A) of the normalized microarray data demonstrates excellent clustering of the two distinct cell populations, indicating distinct and reproducible mRNA expression profiles amongst biological replicates. Transcripts with significant differential expression between the miR-6891-5p inhibition and control sample groups were identified. One hundred four up-regulated and 99 down-regulated transcripts were observed within the miR-6891-5p inhibition sample group as compared to the control group, using a fold change cutoff of >2 and a false discovery rate (FDR) cutoff of 0.05 (FIG. 3B; Table 51 and Table S2 respectively). Since miRNA are known to bind and down regulate the expression of targeted mRNA transcripts, only those transcripts that were identified as up-regulated in the miR-6891-5p inhibition sample group were considered to be putative direct targets of miR-6891-5p (Table S1), whereas the set of down-regulated transcripts may be related to indirect effects of miR-6891-5p inhibition (Table S2). The potential binding sites of miR-6891-5p within the 104 up-regulated transcripts were identified using an in silico miRNA target prediction algorithm. Among the 104 empirically identified putative targets of miR-6891-5p, 61 (˜58%) were found to harbor a computationally predicted miRNA binding site for miR-6891-5p (Table S1).

Functional analysis of differentially expressed transcripts was performed by determining the enriched gene ontology (GO) biological processes of up regulated and down-regulated transcripts (Tables S3A-B). Significantly up-regulated transcripts were found to be involved in numerous immunological processes including leukocyte and mast cell activation (GIMAP5, EGR1, NDRG1 and LCP2) and a variety of cellular processes including T-cell antigen receptor mediated signaling (LCP2) and T-cell quiescence (GIMAP5). Also amongst the significant up-regulated transcripts are 11 DNA binding proteins and transcription factors (FOS, EGR1, LEF1, TP63, HIST1H2AG, ZFHX4, ZNF730, ZNF83) including the transcriptional repressor genes SNAI2, PCGF2 and ZNF253. Significantly down regulated transcripts, following miR-6891-5p inhibition, are involved in numerous immunological processes including cytokine production (FCER1G, HMOX1, IFNG, IL10, NFATC2, SIRT1, TSPAN6), regulation of B cell mediated immunity (FCER1G, IFNG, IL10), inflammation (CCR1, CXCL10, FCER1G, HMOX1, IL10, PNMA1, PPARG) and the immunoglobulin mediated immune response (FCER1G, IFNG, IL10).

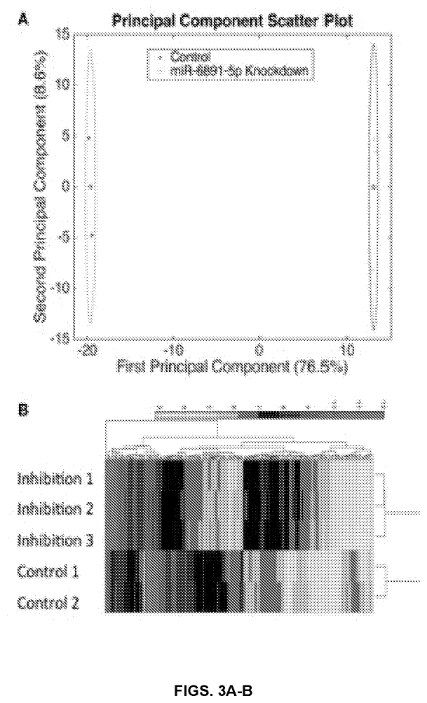

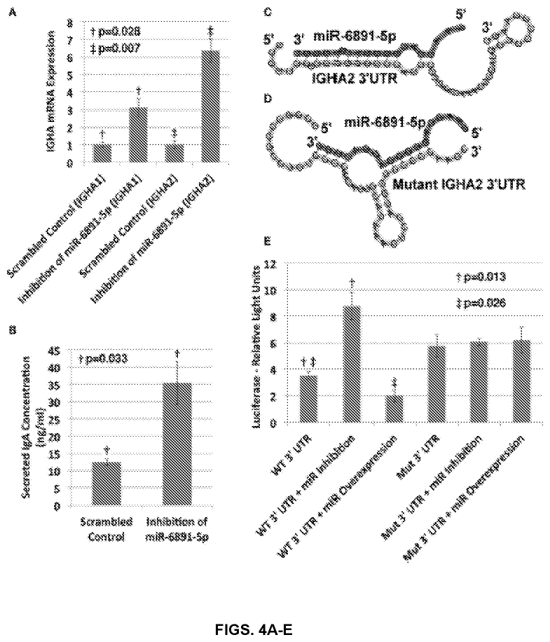

miR-6891-5p Mediated Regulation of IgA. The IgA heavy chain encoding transcript, was among the most significantly up regulated transcripts following inhibition of miR-6891-5p identified from the microarray analysis (8.5-fold-change, FDR=0.02). To study the role of miR-6891-5p on the abundance of both the IgA mRNA transcript and secreted IgA protein, IgA secreting COX cells were transduced with a lentivirus expressing either the antisense of miR-6891-5p (miR-6891-5p inhibition) or a scrambled antisense sequence of miR-6891-5p (control). Inhibition of miR-6891-5p within COX cells significantly increased the abundance of both the IGHA1 and IGHA2 mRNA transcripts (p=0.028 and p=0.007 respectively) (FIG. 4A) and secreted IgA protein (p=0.033) (FIG. 4B) compared to cells transduced with the scrambled control. These findings demonstrate that miR-6891-5p inhibits the expression of both IGHA1 and IGHA2. In silico molecular modeling of both IGHA1 and IGHA2 transcripts reveals an energetically favorable binding site of miR-6891-5p on the 3′UTR of IGHA1 that is 100% conserved within the 3′UTR of the IGHA2 transcript, suggesting that miR-6891-5p may bind and regulate the expression of both transcripts. The identified non-canonical heteroduplex contains limited base pairing between the miRNA seed region (positions 2-7 of the 5′ end) and the conserved 3′UTR sequence of the IGHA1 and IGHA2 transcripts (FIG. 4C).

To validate functional targeting of the modeled miR-6891-5p binding site within the conserved 3′UTR sequences of both IGHA1 and IGHA2, the UTR sequence was fused to a plasmid-based luciferase reporter and transfected into HEK293T cells. HEK293T cells express miR-6891-5p but not IgA and thus provide a cell model system to study IGHA 3′UTR targeting without competitive binding from endogenously expressed IGHA mRNA. These cells were also transfected with either the miR-6891-5p antisense expression plasmid to inhibit endogenously expressed miR-6891-5p (miR inhibition) or a plasmid expressing miR-6891-5p to increase the level of the endogenously expressed miR-6891-5p (miR overexpression). Inhibition of miR-6891-5p significantly increased luciferase activity (p=0.013), whereas overexpression of miR-6891-5p significantly attenuated luciferase activity (p=0.026). Further validation of the binding site was performed by mutating the 3′UTR sequence underlying the binding site of miR-6891-5p (FIG. 4D) and fusing it to a plasmid-based luciferase reporter, which was then transfected into HEK293T cells. These cells were also transfected with either the miR-6891-5p antisense expression plasmid to inhibit endogenously expressed miR-6891-5p or a plasmid expressing miR-6891-5p to increase the level of the endogenously expressed miR-6891-5p. In contrast to the wild-type 3′UTR luciferase experiments, no modulation of miR-6891-5p (inhibition or overexpression) was able to affect luciferase activity (FIG. 4E), indicating that miR-6891-5p was unable to bind the mutant 3′UTR sequence. Together, these results suggest direct miR-6891-5p targeting on the 3′UTR of both the IGHA1 and IGHA2 transcripts.

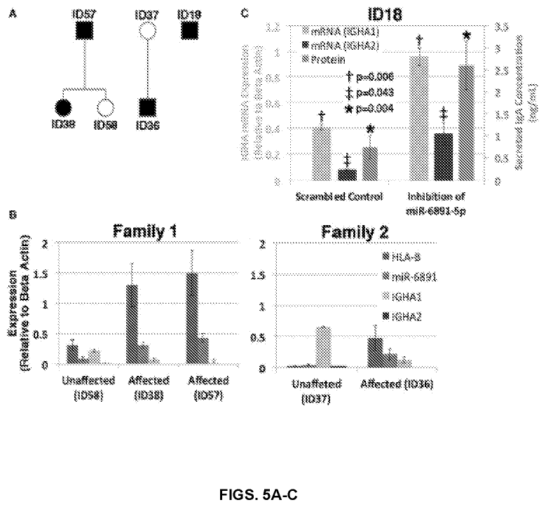

Implications for Selective IgA Deficiency. Given these findings, the inventors investigated the putative role of miR-6891-5p on the expression and secretion of IgA within B-LCLs obtained from two familial cohorts, consisting of individuals affected by selective IgA deficiency and unaffected relatives (FIG. 5A). In order to design effective qPCR primers that amplify the HLA-B mRNA transcripts of each individual, high resolution HLA genotyping was performed on all affected and unaffected individuals for eight HLA loci (Table S4). Phased MHC haplotypes were inferred from related individuals using the family pedigree when available (ID57, ID58, ID38, ID37 and ID36) or from common MHC haplotypes otherwise (ID18). Expression of HLA-B, miR-6891-5p, IGHA1 and IGHA2 was quantified by sequence specific qPCR primers (FIG. 5B). The inventors observe that IGHA1 is the primarily expressed heavy chain transcript of IgA across all individuals and demonstrate an inverse correlation between miR-6891-5p expression and IGHA1 expression (Pearson correlation −0.87), as well as a strong correlation between HLA-B and miR-6891-5p expression (Pearson correlation 0.96), across all patient samples. Both families showed increased expression of both HLA-B (ID36/ID37=18.6×; ID38/1D58=4.2×; ID57/1D58=4.9×) and miR-6891-5p (ID36/1D37=5.3×; ID38/ID58=3.5×; ID57/1D58=16.8×). In all cases miR-6891-5p expression was found to be less than that of the host gene, HLA-B. Additionally, inhibition of miR-6891-5p, in an IgA deficient cell line (ID18) led to a significant increase (˜3×) in both IGHA1 and IGHA2 transcript abundance (p=0.006 and p=0.043 respectively) as well as a significant increase in the concentration of secreted IgA protein (p=0.004) (FIG. 5C).

TABLE S1*

| Ensemble Gene ID | Gene Symbol | Fold Change | FDR |
|---|---|---|---|
| ENSG00000226777 | KIAA0125 | 22.7 | 1.2E−02 |
| ENSG00000211890 | IGHA2 | 8.5 | 2.0E−02 |
| ENSG00000186522 | SEPT10 | 7.8 | 3.8E−03 |
| ENSG00000229807 | XIST | 7.5 | 2.0E−03 |
| ENSG00000133124 | IRS4 | 6.4 | 4.5E−03 |
| ENSG00000237438 | CECR7 | 6.3 | 2.4E−02 |
| ENSG00000258667 | HIF1A-AS2 | 6.0 | 7.5E−04 |
| ENSG00000079691 | LRRC16A | 5.9 | 9.8E−04 |
| ENSG00000184258 | CDR1 | 5.6 | 3.2E−02 |
| ENSG00000073282 | TP63 | 5.4 | 2.6E−03 |
| ENSG00000272870 | LOC105377540 | 4.8 | 1.0E−03 |
| ENSG00000134755 | DSC2 | 4.7 | 5.9E−03 |
| ENSG00000225764 | P3H2-AS1 | 4.2 | 1.7E−02 |
| ENSG00000198865 | CCDC152 | 4.2 | 2.8E−02 |
| ENSG00000120738 | EGR1 | 4.2 | 9.8E−03 |
| ENSG00000253882 | LOC154761 | 4.0 | 1.3E−02 |
| ENSG00000239445 | ST3GAL6-AS1 | 3.9 | 1.7E−03 |
| ENSG00000019549 | SNAI2 | 3.8 | 3.9E−02 |
| ENSG00000261409 | N/A | 3.8 | 3.8E−02 |
| ENSG00000102024 | PLS3 | 3.8 | 1.2E−02 |
| ENSG00000199879 | RNU1-5 | 3.7 | 4.8E−02 |
| ENSG00000222701 | RNY4P7 | 3.6 | 3.9E−02 |
| ENSG00000249096 | LOC102723766 | 3.4 | 4.3E−03 |
| ENSG00000255693 | FLJ41278 | 3.2 | 3.4E−02 |
| ENSG00000236591 | LOC105378047 | 3.2 | 4.1E−03 |
| ENSG00000008311 | AASS | 3.2 | 1.2E−02 |
| ENSG00000253661 | ZFHX4-AS1 | 3.2 | 4.2E−02 |
| ENSG00000228639 | LOC102723505 | 3.1 | 1.3E−02 |
| ENSG00000064225 | ST3GAL6 | 3.1 | 1.9E−02 |
| ENSG00000198780 | FAM169A | 3.1 | 3.1E−02 |
| ENSG00000253140 | LOC105375822 | 3.0 | 3.2E−02 |
| ENSG00000023171 | GRAMD1B | 2.9 | 2.8E−02 |
| ENSG00000256594 | LOC374443 | 2.9 | 1.1E−02 |
| ENSG00000182732 | RGS6 | 2.9 | 2.2E−02 |
| ENSG00000267121 | LOC339192 | 2.8 | 1.3E−02 |
| ENSG00000043462 | LCP2 | 2.7 | 6.3E−03 |
| ENSG00000252847 | RNU2-46P | 2.7 | 1.8E−02 |
| ENSG00000089057 | SLC23A2 | 2.7 | 4.2E−03 |
| ENSG00000196668 | LINC00173 | 2.7 | 3.4E−02 |
| ENSG00000229671 | LINC01150 | 2.7 | 1.9E−02 |
| ENSG00000211772 | TRBV3-1 | 2.7 | 3.7E−03 |
| ENSG00000264468 | MIR4520-1 | 2.7 | 4.3E−02 |
| ENSG00000182621 | PLCB1 | 2.7 | 5.0E−02 |
| ENSG00000006659 | LGALS14 | 2.7 | 4.1E−03 |
| ENSG00000183850 | ZNF730 | 2.7 | 4.3E−02 |
| ENSG00000170379 | FAM115C | 2.6 | 2.8E−02 |
| ENSG00000186352 | ANKRD37 | 2.6 | 1.5E−02 |
| ENSG00000090376 | IRAK3 | 2.5 | 1.2E−02 |
| ENSG00000160856 | FCRL3 | 2.5 | 2.1E−02 |
| ENSG00000257027 | N/A | 2.5 | 5.9E−03 |
| ENSG00000210195 | MT-TT | 2.5 | 3.1E−02 |
| ENSG00000170345 | FOS | 2.5 | 3.1E−02 |
| ENSG00000196329 | GIMAP5 | 2.5 | 1.3E−03 |
| ENSG00000186810 | CXCR3 | 2.5 | 1.9E−02 |
| ENSG00000181074 | OR52N4 | 2.4 | 2.6E−02 |
| ENSG00000078269 | SYNJ2 | 2.4 | 4.6E−02 |
| ENSG00000247095 | MIR210HG | 2.4 | 8.8E−03 |
| ENSG00000075213 | SEMA3A | 2.4 | 2.0E−02 |
| ENSG00000138185 | ENTPD1 | 2.4 | 4.4E−02 |
| ENSG00000095637 | SORBS1 | 2.4 | 2.7E−02 |
| ENSG00000253047 | N/A | 2.4 | 7.6E−03 |
| ENSG00000091656 | ZFHX4 | 2.4 | 3.7E−02 |
| ENSG00000115318 | LOXL3 | 2.4 | 1.1E−02 |
| ENSG00000150991 | UBC | 2.4 | 2.4E−03 |
| ENSG00000137507 | LRRC32 | 2.4 | 4.9E−02 |
| ENSG00000196747 | HIST1H2AG | 2.4 | 3.4E−02 |
| ENSG00000104419 | NDRG1 | 2.3 | 3.4E−02 |
| ENSG00000213988 | ZNF253 | 2.3 | 4.8E−02 |
| ENSG00000111674 | ENO2 | 2.3 | 4.9E−03 |
| ENSG00000200972 | RNU5A | 2.3 | 1.3E−02 |
| ENSG00000244620 | N/A | 2.3 | 1.3E−02 |
| ENSG00000114268 | PFKFB4 | 2.3 | 5.0E−02 |
| ENSG00000101336 | HCK | 2.3 | 7.9E−03 |
| ENSG00000050030 | KIAA2022 | 2.3 | 4.5E−02 |
| ENSG00000180543 | TSPYL5 | 2.3 | 9.7E−03 |
| ENSG00000163564 | PYHIN1 | 2.3 | 2.3E−02 |
| ENSG00000154760 | SLFN13 | 2.2 | 2.5E−02 |
| ENSG00000084710 | EFR3B | 2.2 | 6.6E−03 |
| ENSG00000257345 | LOC105369906 | 2.2 | 4.3E−02 |
| ENSG00000175265 | GOLGA8B | 2.2 | 3.7E−02 |
| ENSG00000251259 | N/A | 2.2 | 4.6E−02 |
| ENSG00000245694 | CRNDE | 2.2 | 2.8E−02 |
| ENSG00000255733 | IFNG-AS1 | 2.2 | 1.3E−02 |
| ENSG00000252026 | RNU6-1262P | 2.2 | 4.7E−02 |
| ENSG00000232445 | LOC101927746 | 2.2 | 2.4E−02 |
| ENSG00000196968 | FUT11 | 2.2 | 3.2E−03 |
| ENSG00000161249 | DMKN | 2.2 | 1.7E−02 |
| ENSG00000173933 | RBM4 | 2.2 | 3.4E−02 |
| ENSG00000101311 | FERMT1 | 2.1 | 4.6E−02 |
| ENSG00000273727 | N/A | 2.1 | 6.8E−03 |
| ENSG00000059804 | SLC2A3 | 2.1 | 7.7E−03 |
| ENSG00000075826 | SEC31B | 2.1 | 1.8E−02 |
| ENSG00000198089 | SFI1 | 2.1 | 2.4E−02 |
| ENSG00000202408 | RNU1-5 | 2.1 | 5.0E−02 |
| ENSG00000138795 | LEF1 | 2.1 | 2.3E−02 |
| ENSG00000235823 | OLMALINC | 2.1 | 2.6E−02 |
| ENSG00000133328 | HRASLS2 | 2.1 | 3.0E−02 |
| ENSG00000179144 | GIMAP7 | 2.1 | 4.2E−02 |
| ENSG00000152256 | PDK1 | 2.1 | 1.6E−03 |
| ENSG00000277258 | PCGF2 | 2.1 | 9.1E−03 |
| ENSG00000007944 | MYLIP | 2.1 | 3.2E−02 |
| ENSG00000236352 | N/A | 2.0 | 4.0E−02 |
| ENSG00000153930 | ANKFN1 | 2.0 | 4.2E−02 |
| ENSG00000167766 | ZNF83 | 2.0 | 2.5E−02 |

*Significantly up-regulated transcripts identified from microarray analysis following miR-6891 inhibition (HSA miR-6891-5p inhibition vs. control samples). Identified genes are putative, direct targets of HSA-miR-6891-5p. High confidence putative targets are shown in bold and additionally contain a predicted HSA-miR-6891-5p binding site within the 3′ UTR of the indicated gene as identified by in silico miRNA target prediction.

TABLE S2**

| Ensemble Gene ID | Gene Symbol | Fold Change | FDR |
|---|---|---|---|
| ENSG00000131016 | AKAP12 | −5.7 | 2.7E−02 |
| ENSG00000140450 | ARRDC4 | −5.5 | 6.3E−04 |
| ENSG00000253874 | IGLVIV-66-1 | −4.3 | 4.0E−02 |
| ENSG00000104722 | NEFM | −4.2 | 3.5E−04 |
| ENSG00000174827 | PDZK1 | −4.2 | 4.2E−02 |
| ENSG00000156689 | GLYATL2 | −4.1 | 2.5E−02 |
| ENSG00000170100 | ZNF778 | −4.0 | 2.0E−02 |
| ENSG00000169245 | CXCL10 | −3.9 | 1.8E−02 |
| ENSG00000269586 | CT45A10 | −3.8 | 9.4E−03 |
| ENSG00000278705 | HIST1H4B | −3.8 | 3.0E−02 |
| ENSG00000005249 | PRKAR2B | −3.7 | 3.0E−02 |
| ENSG00000117009 | KMO | −3.6 | 1.5E−02 |
| ENSG00000130956 | HABP4 | −3.5 | 8.2E−03 |
| ENSG00000135052 | GOLM1 | −3.3 | 2.1E−03 |
| ENSG00000211676 | IGLJ2 | −3.3 | 1.1E−02 |
| ENSG00000138760 | SCARB2 | −3.2 | 1.7E−02 |
| ENSG00000249049 | N/A | −3.2 | 2.0E−02 |
| ENSG00000226562 | CYP4F26P | −3.1 | 4.8E−02 |
| ENSG00000126010 | GRPR | −3.1 | 5.8E−03 |
| ENSG00000103942 | HOMER2 | −3.0 | 2.1E−02 |
| ENSG00000273118 | LOC105373862 | −3.0 | 4.5E−02 |
| ENSG00000131470 | PSMC3IP | −3.0 | 3.1E−03 |
| ENSG00000137941 | TTLL7 | −3.0 | 2.2E−02 |
| ENSG00000137942 | FNBP1L | −2.9 | 2.4E−02 |
| ENSG00000238648 | TRI-TAT2-3 | −2.9 | 3.7E−02 |
| ENSG00000211648 | IGLV1-47 | −2.9 | 4.2E−02 |
| ENSG00000165912 | PACSIN3 | −2.9 | 1.1E−03 |
| ENSG00000166342 | NETO1 | −2.8 | 6.2E−03 |
| ENSG00000102241 | HTATSF1 | −2.8 | 7.1E−03 |
| ENSG00000263961 | C1ORF186 | −2.8 | 4.7E−03 |
| ENSG00000154059 | IMPACT | −2.8 | 8.5E−03 |
| ENSG00000158869 | FCER1G | −2.8 | 3.9E−02 |
| ENSG00000170500 | LONRF2 | −2.8 | 2.5E−02 |
| ENSG00000132970 | WASF3 | −2.7 | 3.1E−02 |
| ENSG00000111537 | IFNG | −2.7 | 2.6E−02 |
| ENSG00000277586 | NEFL | −2.7 | 4.8E−03 |
| ENSG00000140465 | CYP1A1 | −2.7 | 2.3E−02 |
| ENSG00000238244 | GABARAPL3 | −2.7 | 3.3E−02 |
| ENSG00000087191 | PSMC5 | −2.7 | 7.5E−03 |
| ENSG00000135698 | MPHOSPH6 | −2.7 | 5.0E−03 |
| ENSG00000137267 | TUBB2A | −2.7 | 3.3E−02 |
| ENSG00000000003 | TSPAN6 | −2.6 | 2.5E−02 |
| ENSG00000132465 | IGJ | −2.6 | 5.7E−03 |
| ENSG00000072133 | RPS6KA6 | −2.5 | 3.5E−02 |
| ENSG00000100557 | C14ORF105 | −2.5 | 2.5E−03 |
| ENSG00000170846 | LOC93622 | −2.5 | 2.7E−03 |
| ENSG00000214941 | ZSWIM7 | −2.5 | 2.2E−02 |
| ENSG00000169957 | ZNF768 | −2.5 | 1.5E−02 |
| ENSG00000088256 | GNA11 | −2.5 | 9.8E−03 |
| ENSG00000136634 | IL10 | −2.5 | 8.8E−03 |
| ENSG00000110090 | CPT1A | −2.5 | 1.1E−02 |
| ENSG00000173212 | C1ORF161 | −2.4 | 1.8E−02 |
| ENSG00000100292 | HMOX1 | −2.4 | 3.9E−03 |
| ENSG00000148468 | FAM171A1 | −2.4 | 4.0E−02 |
| ENSG00000169857 | AVEN | −2.4 | 2.7E−02 |
| ENSG00000198648 | STK39 | −2.4 | 2.3E−02 |
| ENSG00000082458 | DLG3 | −2.4 | 3.1E−02 |
| ENSG00000163823 | CCR1 | −2.4 | 3.1E−02 |
| ENSG00000137673 | MMP7 | −2.4 | 1.5E−02 |
| ENSG00000182534 | MXRA7 | −2.3 | 4.4E−02 |
| ENSG00000198283 | OR5B21 | −2.3 | 3.8E−02 |
| ENSG00000185900 | SGK196 | −2.3 | 1.9E−03 |
| ENSG00000138678 | AGPAT9 | −2.3 | 1.2E−02 |
| ENSG00000132170 | PPARG | −2.3 | 2.3E−02 |
| ENSG00000108830 | RND2 | −2.3 | 2.1E−02 |
| ENSG00000175857 | GAPT | −2.3 | 3.6E−02 |
| ENSG00000198729 | PPP1R14C | −2.3 | 4.1E−02 |
| ENSG00000213886 | UBD | −2.3 | 2.9E−02 |
| ENSG00000125869 | C20ORF103 | −2.3 | 1.1E−02 |
| ENSG00000183723 | CMTM4 | −2.2 | 3.0E−02 |
| ENSG00000162627 | SNX7 | −2.2 | 1.9E−02 |
| ENSG00000169914 | OTUD3 | −2.2 | 4.3E−02 |
| ENSG00000179010 | MRFAP1 | −2.2 | 1.3E−02 |
| ENSG00000167674 | HDGFRP2 | −2.2 | 7.4E−03 |
| ENSG00000021355 | SERPINB1 | −2.2 | 9.8E−03 |
| ENSG00000181191 | PJA1 | −2.2 | 1.4E−02 |
| ENSG00000188558 | OR2G6 | −2.2 | 1.8E−02 |
| ENSG00000115109 | EPB41L5 | −2.2 | 4.0E−02 |
| ENSG00000129250 | KIF1C | −2.2 | 1.8E−03 |
| ENSG00000117174 | ZNHIT6 | −2.2 | 2.4E−02 |
| ENSG00000211972 | IGHV3-66 | −2.1 | 4.0E−02 |
| ENSG00000235493 | N/A | −2.1 | 3.6E−02 |
| ENSG00000203661 | OR2T5 | −2.1 | 4.4E−02 |
| ENSG00000183624 | C3ORF37 | −2.1 | 8.4E−03 |
| ENSG00000114446 | IFT57 | −2.1 | 2.3E−03 |
| ENSG00000180015 | LOC285442 | −2.1 | 4.3E−02 |
| ENSG00000169435 | RASSF6 | −2.1 | 6.9E−03 |
| ENSG00000111275 | ALDH2 | −2.1 | 2.3E−02 |
| ENSG00000169750 | RAC3 | −2.1 | 4.1E−03 |
| ENSG00000102390 | CXORF26 | −2.1 | 1.9E−02 |
| ENSG00000183688 | FAM101B | −2.0 | 1.2E−02 |
| ENSG00000260943 | LOC101930164 | −2.0 | 3.1E−02 |
| ENSG00000176903 | PNMA1 | −2.0 | 2.3E−02 |
| ENSG00000067533 | RRP15 | −2.0 | 2.1E−02 |
| ENSG00000160633 | SAFB | −2.0 | 3.0E−03 |
| ENSG00000101096 | NFATC2 | −2.0 | 3.4E−02 |
| ENSG00000211677 | IGLV2-11 | −2.0 | 1.1E−02 |
| ENSG00000096717 | SIRT1 | −2.0 | 2.7E−02 |
| ENSG00000188641 | DPYD | −2.0 | 2.8E−02 |

**Significantly down-regulated transcripts identified from microarray analysis (miR-6891-5p inhibition vs. control samples). Identified mRNA transcripts presumably reflect indirect effects of miR-6891-5p inhibition on lymphoblastoid cells rather than direct miRNA binding of listed transcripts.

TABLE S3A\hat{ }
FUNCTIONAL ENRICHMENT OF SIGNIFICANT UPREGULATED
TRANSCRIPTS (Inhibition vs. Control)

| Gene Ontology Term | P-Value | Fold Enrichment |
|---|---|---|
| mesoderm development | 1.5E−03 | 1.7E+01 |
| ectoderm and mesoderm interaction | 6.2E−03 | 3.1E+02 |
| myeloid leukocyte activation | 8.9E−03 | 2.1E+01 |
| cell activation | 1.2E−02 | 5.5E+00 |
| positive regulation of macromolecule biosynthetic process | 1.5E−02 | 3.4E+00 |
| positive regulation of cellular biosynthetic process | 1.8E−02 | 3.2E+00 |
| positive regulation of biosynthetic process | 1.9E−02 | 3.2E+00 |
| response to organic substance | 2.3E−02 | 3.1E+00 |
| mast cell activation | 2.5E−02 | 7.9E+01 |
| positive regulation of transcription from RNA polymerase II promoter | 2.7E−02 | 4.2E+00 |
| positive regulation of transcription | 3.0E−02 | 3.3E+00 |
| monosaccharide metabolic process | 3.1E−02 | 5.7E+00 |
| positive regulation of gene expression | 3.3E−02 | 3.2E+00 |
| leukocyte activation | 3.9E−02 | 5.2E+00 |
| positive regulation of nucleobase, nucleoside, nucleotide and nucleic | 4.3E−02 | 3.0E+00 |
| acid metabolic process | | |
| learning or memory | 4.6E−02 | 8.5E+00 |
| positive regulation of macromolecule metabolic process | 4.7E−02 | 2.6E+00 |
| positive regulation of nitrogen compound metabolic process | 4.8E−02 | 2.9E+00 |
| negative regulation of transcription from RNA polymerase II promoter | 4.9E−02 | 4.7E+00 |

TABLE S3B\hat{ }
FUNCTIONAL ENRICHMENT OF SIGNIFICANT DOWNREGULATED
TRANSCRIPTS (Inhibition vs. Control)

| Gene Ontology Term | P-Value | Fold Enrichment |
|---|---|---|
| regulation of production of molecular mediator of immune response | 8.6E−04 | 2.1E+01 |
| regulation of membrane protein ectodomain proteolysis | 1.6E−03 | 5.0E+01 |
| regulation of leukocyte mediated immunity | 2.7E−03 | 1.4E+01 |
| regulation of cytokine production during immune response | 3.0E−03 | 3.6E+01 |
| heterocycle catabolic process | 5.3E−03 | 1.1E+01 |
| regulation of B cell mediated immunity | 5.3E−03 | 2.7E+01 |
| regulation of immunoglobulin mediated immune response | 5.3E−03 | 2.7E+01 |
| behavior | 5.5E−03 | 3.7E+00 |
| taxis | 6.2E−03 | 6.7E+00 |
| chemotaxis | 6.2E−03 | 6.7E+00 |
| regulation of cytokine production | 9.5E−03 | 5.9E+00 |
| regulation of immune effector process | 1.1E−02 | 8.5E+00 |
| negative regulation of cytokine production | 1.3E−02 | 1.7E+01 |
| response to organic cyclic substance | 1.8E−02 | 7.1E+00 |
| receptor biosynthetic process | 1.8E−02 | 1.1E+02 |
| regulation of mast cell cytokine production | 1.8E−02 | 1.1E+02 |
| positive regulation of MHC class II biosynthetic process | 2.3E−02 | 8.6E+01 |
| regulation of gene-specific transcription | 2.4E−02 | 6.4E+00 |
| regulation of protein catabolic process | 2.4E−02 | 1.2E+01 |
| regulation of proteolysis | 2.5E−02 | 1.2E+01 |
| organic ether metabolic process | 2.5E−02 | 1.2E+01 |
| regulation of lymphocyte mediated immunity | 2.5E−02 | 1.2E+01 |
| response to alkaloid | 2.5E−02 | 1.2E+01 |
| regulation of adaptive immune response based on somatic recombination of | 2.6E−02 | 1.2E+01 |
| immune receptors built from immunoglobulin superfamily domains | | |
| regulation of cell proliferation | 2.7E−02 | 2.5E+00 |
| regulation of cellular localization | 2.7E−02 | 4.3E+00 |
| regulation of adaptive immune response | 2.7E−02 | 1.2E+01 |
| positive regulation of chemokine biosynthetic process | 3.2E−02 | 6.1E+01 |
| white fat cell differentiation | 3.2E−02 | 6.1E+01 |
| regulation of cellular catabolic process | 3.2E−02 | 1.1E+01 |
| regulation of MHC class II biosynthetic process | 3.6E−02 | 5.4E+01 |
| response to hyperoxia | 3.6E−02 | 5.4E+01 |
| locomotory behavior | 3.7E−02 | 3.9E+00 |
| immune response | 3.8E−02 | 2.5E+00 |
| negative regulation of multicellular organismal process | 3.9E−02 | 5.2E+00 |
| positive regulation of membrane protein ectodomain proteolysis | 4.1E−02 | 4.8E+01 |
| regulation of chemokine biosynthetic process | 4.5E−02 | 4.3E+01 |
| regulation of myeloid leukocyte mediated immunity | 4.5E−02 | 4.3E+01 |
| regulation of cytokine biosynthetic process | 4.5E−02 | 8.7E+00 |
| regulation of inflammatory response | 4.7E−02 | 8.5E+00 |

\hat{ }Gene ontology (GO) functional enrichment of significant, differentially expressed transcripts identifiedfrom microarray analysis. GO biological processes (level 4) were annotated using a p value cutoff of 0.05.

TABLE S4*

| Sample | | | Disease | | | | HLA- | HLA- | HLA- | HLA- | HLA- |
|---|---|---|---|---|---|---|---|---|---|---|---|
| ID | Family | Relationship | Status | HLA-A | HLA-B | HLA-C | DRB1 | DQA1 | DQB1 | DPA1 | DPB1 |
| ID57 | 1 | Father | Affected | 01:01:01 | 08:01:01 | 07:01:01 | 03:01:01 | 05:01:01 | 02:01:01 | 02:01:02 | 01:01:01 |
| | | Father | | 02:01:01 | 40:02:01 | 15:02:01 | 04:01:01 | 03:03:01 | 03:01:01 | 01:03:01 | 04:02:01 |
| ID58 | 1 | Daughter | Unaffected | 01:01:01 | 08:01:01 | 07:01:01 | 03:01:01 | 05:01:01 | 02:01:01 | 02:01:02 | 01:01:01 |
| | | Daughter | | 29:02:01 | 44:03:01 | 16:01:01 | 07:01:01 | 02:01:01 | 02:02:01 | 02:02:02 | 01:01:01 |
| ID38 | 1 | Daughter | Affected | 01:01:01 | 08:01:01 | 07:01:01 | 03:01:01 | 05:01:01 | 02:01:01 | 02:01:02 | 01:01:01 |
| | | Daughter | | 02:01:01 | 40:01:02 | 03:04:01 | 13:01:01 | 01:03:01 | 06:03:01 | 02:01:01 | 02:01:02 |
| ID37 | 2 | Mother | Unaffected | 26:01:01 | 38:01:01 | 12:03:01 | 04:02:01 | 03:01:01 | 03:02:01 | 01:03:01 | 04:01:01 |
| | | Mother | | 24:02:01 | 08:01:01 | 07:01:01 | 03:01:01 | 05:01:01 | 02:01:01 | 01:03:01 | 04:01:01 |
| ID36 | 2 | Son | Affected | 26:01:01 | 38:01:01 | 12:03:01 | 04:02:01 | 03:01:01 | 03:02:01 | 01:03:01 | 04:01:01 |
| | | Son | | 32:01:01 | 07:05:01 | 04:01:01 | 10:01:01 | 01:05:01 | 05:01:01 | 01:03:01 | 04:01:01 |
| ID18 | 3 | N/A | Affected | 01:01:01 | 08:01:01 | 07:01:01 | 03:01:01 | 05:01:01 | 02:01:01 | 01:03:01 | 04:01:01 |
| | | N/A | | 02:01:01 | 35:01:01 | 04:01:01 | 04:02:01 | 03:01:01 | 03:02:01 | 01:03:01 | 04:01:01 |

*High resolution HLA genotyping results of B-LCLs obtained from patients with selective IgA deficiency and unaffected, related family members. All samples were obtained from the Coriell Biorepository. Phased MHC haplotypes were inferred from related individuals when available (ID57, ID58, ID38, ID37 and ID36) and based upon common MHC haplotypes otherwise (ID18).

### Example 3—Discussion

HLA molecules are best known for their role in the antigen-specific immune response and in differentiating self from non-self. However, this research suggests a novel regulatory role of the HLA-B gene mediated by a co-transcribed miRNA, miR-6891-5p, encoded within intron 4 of the HLA-B transcript (Ladewig et al., 2012). This analysis reveals that miR-6891-5p is 100% conserved across every annotated full-length HLA-B allele, while miR-6891-3p contains two polymorphic locations, including one within the seed region. The inventors' previous research quantifying class I HLA allele sequence diversity demonstrates that intron 4 of HLA-B is the most conserved intron among class I HLA genes (Clark et al., 2016b). The sequence conservation of miR-6891-5p amongst HLA-B alleles, as well as amongst other non-human primates suggests that this miRNA plays an important regulatory role and forms the basis for the functional study of miR-6891-5p.

This functional study of miR-6891-5p within B-LCLs suggests that miR-6891-5p regulates the expression of nearly 200 transcripts, which are involved in numerous immunological processes. Since miRNAs are known to attenuate the post-transcriptional expression of targeted transcripts, inhibition of miR-6891-5p would be expected to up-regulate the expression of directly targeted transcripts. However, because miR-6891-5p inhibition was found to up-regulate the expression of several transcription factors, (all of which contain a computationally predicted miR-6891-5p binding site) it is possible that many of the observed differentially expressed transcripts may result from indirect, downstream effects of miR-6891-5p inhibition that are mediated by targeted transcription factors. Because three of the identified targeted transcription factors are known transcriptional repressors (SNAI2, PCGF2 and ZNF253), it is likely that the up regulation of these repressors following miR-6891-5p inhibition would attenuate the transcription of numerous genes, resulting in the observed down-regulation of numerous transcripts following miR-6891-5p inhibition. Similarly, the observed up-regulation of transcriptional activators (LEF1, EGR1, TP63 and FOS) following miR-6891-5p inhibition, may up-regulate the transcription of numerous genes that are not direct targets of miR-6891-5p and may partially explain the observed up-regulation of genes that do not harbor a computationally predicted binding site of miR-6891-5p. Together these data suggest that miR-6891-5p not only regulates the post-transcriptional expression of directly targeted transcripts, but may also modulate the transcription of numerous other genes indirectly, through miR-6891-5p mediated translational repression of targeted transcriptional activators and/or repressors. These results suggest an important physiological role of miR-6891-5p within B-LCLs. The ubiquitous expression of HLA-B also suggests that miR-6891-5p may play a broader role in a variety of tissues and cellular phenotypes, and is the subject of ongoing research.

Upon miR-6891-5p inhibition, transcripts encoding the heavy chain of IgA were found to be amongst the top identified up-regulated transcripts. This particular target of miR-6891-5p was selected for further validation since immunoglobulin production is a key function of plasma cells and no miRNA has been shown to directly bind and regulate immunoglobulin expression, although miR-155 has been shown to indirectly influence immunoglobulin expression through regulation of B cell differentiation and maturation (Vigorito et al., 2007). Despite the lack of a predicted miR-6891-5p binding site on either the IGHA1 or IGHA2 transcript using current computational miRNA target prediction algorithms (Table S1), the inventors' molecular modeling of the miR-6891-5p, IGHA1 and IGHA2 transcripts reveals an energetically favorable, non-canonical heteroduplex formation, with limited base pairing within the miRNA seed region (traditionally defined as base positions 2-7 of the 5′ end of the mature miRNA) and the identified target site on the 3′UTR of both the IGHA1 and IGHA2 transcripts. Experimental validation of the modeled miR-6891-5p binding site within the 3′UTR sequence by the luciferase reporter assay (including control experiments using a mutated sequence of the miR-6891-5p binding site) indicates miR-6891-5p mediated post-transcriptional regulation of IgA through the modeled, non-canonical interaction with the 3′ UTR of both the IGHA1 and IGHA2 transcripts. Because the binding site of miR-6891-5p on the 3′UTR of both IGHA1 and IGHA2 transcripts is 100% conserved, these results indicate that miR-6891-5p regulates the expression of both transcripts through an interaction within a conserved target site present on the 3′UTR of both transcripts, effectively mediating the post-transcriptional expression of both the IGHA1 and IGHA2 transcripts. Recent research suggests that the existence of non-canonical heteroduplex formations between a miRNA and its target may be more prevalent than previously thought (Helwak et al., 2013). This in turn may lead to false-negative miRNA target predictions by algorithms that rely on a high degree of Watson-crick base complementary between the seed region of a given miRNA and the predicted target site. Together these considerations suggest that the number of significantly up-regulated transcripts following inhibition of miR-6891-5p that harbor a computationally predicted miR-6891-5p binding site (58%) may be an underestimate of the true number of directly targeted transcripts identified by microarray expression analysis following miR-6891-5p inhibition.

The inventors' initial findings led us to investigate the putative role of miR-6891-5p in the pathophysiology of selective IgA deficiency within B-LCLs obtained from affected individuals and unaffected family members. Selective IgA deficiency is the most common form of primary immunodeficiency and is characterized by the dysregulation of IgA synthesis within immature B lymphocytes resulting in diminished levels of IgA in patient serum (Cunningham-Rundles, 2001; Yel, 2010). B-LCLs obtained from affected individuals were found to express significantly increased levels of both HLA-B and miR-6891-5p as compared to unaffected family members. The expression of miR-6891-5p and the host gene, HLA-B, were highly correlated (Pearson 0.96). Consistent with the inventors' previous findings, expression of miR-6891-5p was inversely correlated with IGHA1 and IGHA2 expression (Pearson −0.8 and -0.86 respectively). Abundance of miR-6891-5p was found to be less than that of the host gene, HLA-B, which is consistent with previous findings correlating mirtron and host gene expression (Wen et al., 2015). Inhibition of miR-6891-5p within B-LCLs isolated from a patient with selective IgA deficiency was found to significantly increase the abundance of both IGHA1 and IGHA2 mRNA as well as secreted IgA protein. Although the genetic etiology of the disease remains to be fully elucidated, a recent GWAS study has demonstrated a primary association within the HLA class II region, and an independent association within the HLA class I (HLA-B) and HLA class III region of the MHC, suggesting a complex genetic association resulting from the combined effects of variants spanning the class I, II and III HLA regions (Ferreira et al., 2012). Additionally, the HLA-A*01-B*08-DRB1*0301-DQB1*02 (DR3), HLA-B*14-DRB1*0102-DQB1*05 (DR1) and HLA-B*44-DRB1*0701-DQB1*02 (DR7) MHC haplotypes have all been associated with IgA deficiency, while the HLA-DRB1*1501-DQB1*06 (DR2) MHC haplotype has been shown to confer protection against IgA deficiency (Olerup et al., 1990; Ferreira et al., 2012). Previous research further demonstrates that the prevalence of IgA deficiency amongst HLA-B8-DR3 homozygous individuals ranges between 1.7% (Mohammadi et al., 2010) and ˜13% (Schroeder et al., 1998; Alper et al., 2000). Furthermore, the HLA genotyping of all family members analyzed by the study (affected and unaffected by IgA deficiency) reveals that ¾ of the affected individuals (ID57, ID38, ID18) and all (2/2) of the unaffected individuals are heterozygous for the B8-DR3 haplotype (Table S4), further demonstrating that IgA deficiency likely stems from a number of heterogeneous genetic effects acting in a concerted manner (Yel, 2010). Considering these findings along with the absence of polymorphisms within the miR-6891-5p gene and the observed significantly elevated expression of HLA-B and miR-6891-5p within B-LCLs from patients with selective IgA deficiency, these data suggest a disease model in which the accumulation of miR-6891-5p transcripts may play a role in the pathophysiology of the disease by attenuating expression of IgA. Although the precise mechanism by which this occurs is the subject of ongoing research, it is possible that the primary GWAS signals previously reported by others may result from polymorphisms within an eQTL or other genomic elements present on the associated susceptible MHC haplotypes that result in the increased expression of miR-6891-5p. Thus, it is possible that altered miR-6891-5p expression may be a contributing factor in the pathophysiology of selective IgA deficiency and warrants further study within primary tissue samples from affected individuals.

Our study is the first to describe a functional role of the HLA-B encoded miRNA, miR-6891-5p, and signifies a paradigm shift in the fundamental understanding of the role of the HLA-B gene. The inventors' recent efforts to characterize the miRNA transcriptome of BLCLs, suggest that other HLA genes also encode functional miRNA transcripts (Clark et al., 2016a). Together these works lay the groundwork for further studies investigating the role of HLA encoded miRNAs in regulating transcripts involved in the immune response and other metabolic processes. Previous research demonstrates that 90% of causal autoimmune disease variants are located within non-coding regions of the genome (Farh et al., 2015). Given the ubiquitous expression of class I HLA genes within nearly all nucleated cells, detailed characterization of the regulatory role of HLA encoded miRNAs across various cell types and disease states may reveal interesting new insights offering a potential explanation for some of the reported disease associations within non-coding regions of the MHC. Thus, the current work necessitates additional efforts to better characterize and study the functional role of miRNA transcripts originating from amongst the most complex and under characterized region of the genome, the MHC.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods, and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit and scope of the disclosure as defined by the appended claims.

### IX. REFERENCES

The following references, to the extent that they provide exemplary procedural or other details supplementary to those set forth herein, are specifically incorporated herein by reference.
U.S. Pat. No. 5,844,107
U.S. Pat. No. 5,877,302
U.S. Pat. No. 5,972,900
U.S. Pat. No. 5,972,901
U.S. Pat. No. 6,008,336
U.S. Pat. No. 6,077,835
U.S. Pat. No. 6,200,801
U.S. Pat. No. 6,693,187
U.S. Pat. No. 6,838,283
U.S. Pat. No. 7,067,641
U.S. Publn. 20020150626
U.S. Publn. 20030032615
U.S. Publn. 20030203865
U.S. Publn. 20040048787
Alper et al., Tissue Antigens 56(3), 207-216, 2000.
Ambros, Cell, 113(6):673-676, 2003.
Angel et al., Cell, 49:729, 1987b.
Angel et al., Mol. Cell. Biol., 7:2256, 1987a.
Baichwal et al., In: Gene Transfer, Kucherlapati (Ed.), Plenum Press, NY, 117-48, 1986.
Banerji et al., Cell, 27(2 Pt 1):299-308, 1981.
Banerji et al., Cell, 33(3):729-740, 1983.
Benvenisty and Neshif, Proc. Natl. Acad. Sci. USA, 83(24):9551-9555, 1986.
Berkhout et al., Cell, 59:273-282, 1989.
Blanar et al., EMBO J, 8:1139, 1989.
Bodine et al., EMBO J, 6:2997, 1987.
Bolstad et al., Bioinformatics (Oxford, England) 2003; 19:185-93.
Boshart et al., Cell, 41:521, 1985.
Bosze et al., EMBO J., 5(7):1615-1623, 1986.
Braddock et al., Cell, 58:269, 1989.
Brennecke et al., Cell, 113:25-36, 2003.
Bulla et al., J Virol., 62:1437, 1986.
Calin et al., Proc. Natl. Acd. Sci. USA, 99:15524-15529, 2002.
Campbell and Villarreal, Mol. Cell. Biol., 8:1993, 1988.
Campere and Tilghman, Genes and Dev., 3:537, 1989.
Campo et al., Nature, 303:77, 1983.
Care et al., Nat. Med. 13:613-618, 2007.
Carrington et al. Science, 301(5631):336-338, 2003.
Celander and Haseltine, J. Virology, 61:269, 1987.
Celander et al., J. Virology, 62:1314, 1988.
Chandler et al., Cell, 33:489, 1983.
Chang and Karin, Nature, 410(6824):37-40, 2001.
Chang et al., Nature, 430(7001):785-789, 2004.
Chatterjee et al., Proc. Natl. Acad. Sci. USA, 86:9114, 1989.
Chen and Okayama, Mol. Cell Biol., 7(8):2745-2752, 1987.
Chen et al., Science, 303(5654):83-86, 2004.
Chitnis et al., Molecular Cell, 48:353-64, 2012.
Choi et al., Cell, 53:519, 1988.
Clark et al., Human Immunology 77, Supplement, 38. doi: dx.doi.org/10.1016/j.humimm.2016.07.064, 2016a.
Clark et al., Clin Chem 62(12), 1630-1638. doi: 10.1373/clinchem.2016.260661, 2016b.
Clark et al., Int J Immunogenet 42(6), 413-422. doi: 10.1111/iji.12236, 2015.
Cohen et al. J Cell. Physiol., 5:75, 1987.
Costa et al., Mol. Cell. Biol., 8:81, 1988.
Coupar et al., Gene, 68:1-10, 1988.
Cripe et al., EMBO J., 6:3745, 1987.
Culotta and Hamer, Mol. Cell. Biol., 9:1376, 1989.
Cunningham-Rundles, C. J Clin Immunol 21(5), 303-309, 2001.
Dai et al., Nucleic Acids Research; 33:e175, 2005.
Dandolo et al., J. Virology, 47:55-64, 1983.
De Villiers et al., Nature, 312(5991):242-246, 1984.
Deschamps et al., Science, 230:1174-1177, 1985.
Dubensky et al., Proc. Natl. Acad. Sci. USA, 81:7529-7533, 1984.
Duke et al., Hla 87(3), 141-152. doi: 10.1111/tan.12736, 2016.
Dweep et al., Nature Methods, 12:697, 2015.
Edbrooke et al., Mol. Cell. Biol., 9:1908, 1989.
Edlund et al., Science, 230:912-916, 1985.
EPO 0273085
Farh et al., Nature 518(7539), 337-343. doi: 10.1038/nature13835, 2015.
Fatkin et al. J Clin. Invest., 106(11):1351-1359, 2000.
Fechheimer, et al., Proc Natl. Acad. Sci. USA, 84:8463-8467, 1987.
Feng and Holland, Nature, 334:6178, 1988.
Ferkol et al., FASEB J., 7:1081-1091, 1993.
Ferreira et al., PLoS Genet 8(1), e1002476. doi:10.1371/journal.pgen.1002476, 2012.
Firak and Subramanian, Mol. Cell. Biol., 6:3667, 1986.
Foecking and Hofstetter, Gene, 45(1):101-105, 1986.
Fraley et al., Proc. Natl. Acad. Sci. USA, 76:3348-3352, 1979.
Friedman et al., Genes Devel., 3:1314, 1989.
Friedman et al., Genome Res. 19(1):92-105, 2009.
Friedman, Br. J. Ophthalmol. 88(2):161-163, 2004.
Fujita et al., Cell, 49:357, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gilles et al., Cell, 33:717, 1983.
Gloss et al., EMBO J., 6:3735, 1987.
Godbout et al., Mol. Cell. Biol., 8:1169, 1988.
Goodbourn and Maniatis, Proc. Natl. Acad. Sci. USA, 85:1447, 1988.
Goodbourn et al., Cell, 45:601, 1986.
Gopal, Mol. Cell Biol., 5:1188-1190, 1985.
Gordanpour et al., J Visualized Exp, JoVE, e3874, 2012.
Graham and Van Der Eb, Virology, 52:456-467, 1973.
Greene et al., Immunology Today, 10:272, 1989
Grosschedl and Baltimore, Cell, 41:885, 1985.
Harland and Weintraub, J. Cell Biol., 101(3):1094-1099, 1985.
Haslinger et al., Proc. Natl. Acad. Sci. USA, 82:8572, 1985.
Hauber et al., J Virology, 62:673, 1988.
Helwak et al., Cell 153(3), 654-665. doi: 10.1016/j.cell.2013.03.043, 2013.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Hirochika et al., J. Virol., 61:2599, 1987.
Holbrook et al., Virology, 157:211, 1987.
Horlick and Benfield, Mol. Cell. Biol., 9:2396, 1989.
Horton et al., Nat Rev Genet 5(12), 889-899. doi: 10.1038/nrg1489, 2004.
Horwich et al. J. Virol., 64:642-650, 1990.
Huang et al., Cell, 27:245, 1981.
Huang et al., Nucleic Acids Research, 37:1-13, 2009.
Hug et al., Mol. Cell. Biol., 8:3065, 1988.
Hutvagner et al., PLoS Biol., 2(4):E98, 2004.
Hwang et al., Mol. Cell. Biol., 10:585, 1990.
Imagawa et al., Cell, 51:251, 1987.
Imbra et al., Nature, 323:555, 1986.
Imler et al., Mol. Cell. Biol., 7:2558, 1987.
Imperiale et al., Mol. Cell. Biol., 4:875, 1984.
Irizarry et al., Biostatistics (Oxford, England) 4:249-64; 2003b.
Irizarry et al., Nucleic Acids Research; 31:e15; 2003a.
Jager et al., N Engl. J. Med. 358(24):2606-2617, 2008.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9:3393, 1989.
Jonas and Izaurralde, Nat Rev Genet 16(7), 421-433. doi: 10.1038/nrg3965, 2015.
Kadesch and Berg, Mol. Cell. Biol., 6:2593, 1986.
Kaneda et al., Science, 243:375-378, 1989.
Karin et al., Mol. Cell. Biol., 7:606, 1987.
Katinka et al., Cell, 20:393, 1980.
Kato et al, J. Biol. Chem., 266:3361-3364, 1991.
Kawamoto et al., Mol. Cell. Biol., 8:267, 1988.
Kiledjian et al., Mol. Cell. Biol., 8:145, 1988.
Kiriazis and Kranias, Annu. Rev. Physiol., 62:321-351, 2000.
Klamut et al., Mol. Cell. Biol., 10:193, 1990.
Klein et al., Nature, 327:70-73, 1987.
Koch et al., Mol. Cell. Biol., 9:303, 1989.
Krenz and Robbins, J Am. Coll. Cardiol., 44:2390-2397, 2004.
Kriegler and Botchan, In: Eukaryotic Viral Vectors, Gluzman (Ed.), Cold Spring Harbor: Cold Spring Harbor Laboratory, N Y, 1982.
Kriegler and Botchan, Mol. Cell. Biol., 3:325, 1983a.
Kriegler et al., Cell, 38:483, 1984.
Kriegler et al., Cell, 53:45, 1988.
Kriegler et al., In: Gene Expression, Alan Liss (Ed.), Hamer and Rosenberg, N.Y., 1983b.
Krützfeldt et al., Nature, 438:685-689, 2005.
Kuhl et al., Cell, 50:1057, 1987.
Kunz et al., Nucl. Acids Res., 17:1121, 1989.
Ladewig et al., Genome Research, 22:1634-45, 2012.
Lagos-Quintana et al., Curr Biol. 12:735-739, 2002.
Lagos-Quintana et al., Curr. Biol., 12:735-739, 2002.
Lagos-Quintana et al., Science, 294(5543):853-858, 2001.
Larsen et al., Proc Natl. Acad. Sci. USA., 83:8283, 1986.
Laspia et al., Cell, 59:283, 1989.
Latimer et al., Mol. Cell. Biol., 10:760, 1990.
Lau et al., Science, 294(5543):858-862, 2001.
Lee and Ambros, Science, 294(5543):862-864, 2001.
Lee et al., Biochem. Biophys. Res. Commun. 346(1):83-88, 2006.
Lee et al., Nature, 294:228, 1981.
Levinson et al., Nature, 295:79, 1982.
Lin et al., Mol. Cell. Biol., 10:850, 1990.
Lodish et al., Nat Rev Immunol 8(2), 120-130. doi: 10.1038/nri2252, 2008.
Lowes et al., J. Clin. Invest., 100(9):2315-2324, 1997.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Majors et al., Proc. Natl. Acad. Sci. USA, 80:5866, 1983.
Matsuda & Cepko, Proc. Natl Acad. Sci. USA 101(1):16-22, 2004.
McNeall et al., Gene, 76:81, 1989.
Meister et al., Nature, 431:343-9, 2004.
Miksicek et al., Cell, 46:203, 1986.
Mohammadi et al., J Clin Immunol 30(1), 138-143. doi: 10.1007/s10875-009-9336-2, 2010.
Moreau et al., Nucl. Acids Res., 9:6047, 1981.
Muesing et al., Cell, 48:691, 1987.
Ng et al., Nuc. Acids Res., 17:601, 1989.
Nicolas and Rubinstein, In: Vectors: A survey of molecular cloning vectors and their uses, Rodriguez and Denhardt (Eds), Stoneham: Butterworth, 494-513, 1988.
Nicolau and Sene, Biochim. Biophys. Acta, 721:185-190, 1982.
Nicolau et al., Methods Enzymol., 149:157-176, 1987.
Olerup et al., Nature 347(6290), 289-290. doi: 10.1038/347289a0, 1990.
Omitz et al., Mol. Cell. Biol., 7:3466, 1987.
Ondek et al., EMBO J., 6:1017, 1987.
Palmiter et al., Nature, 300:611, 1982.
Pasquinelli and Ruvkun, Ann. Rev. Cell Dev. Biol., 18:495-513, 2002.
PCT Appln. WO 0071096
Pech et al., Mol. Cell. Biol., 9:396, 1989.
Perales et al., Proc. Natl. Acad. Sci. USA, 91:4086-4090, 1994.
Perez-Stable and Constantini, Mol. Cell. Biol., 10:1116, 1990.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Natl. Acad. Sci. USA, 81:7161-7165, 1984.
Qi et al., Cellular & Molecular Immunology, 3:411-419, 2006.
Queen et al., Cell, 35:741, 1983.
Quinn et al., Mol. Cell. Biol., 9:4713, 1989.
Redondo et al., Science, 247:1225, 1990.
Reisman and Rotter, Mol. Cell. Biol., 9:3571, 1989.
Remington's Pharmaceutical Sciences, 15^{th} ed., pages 1035-1038 and 1570-1580, Mack
Publishing Company, Easton, Pa., 1980.
Resendez et al., Mol. Cell. Biol., 8:4579, 1988.
Ridgeway, In: Vectors: A Survey of Molecular Cloning Vectors and Their Uses, Rodriguez et al. (Eds.), Stoneham: Butterworth, 467-492, 1988.
Ripe et al., Mol. Cell. Biol., 9:2224, 1989.
Rippe, et al., Mol. Cell Biol., 10:689-695, 1990.
Riffling et al., Nuc. Acids Res., 17:1619, 1989.
Robinson et al., Nucleic Acids Res 41 (Database issue), D1222-1227. doi: 10.1093/nar/gks949, 2013.
Rosen et al., Cell, 41:813, 1988.
Rosenfeld et al., N Engl. 1 Med. 355(14):1419-1431, 2006.
Sakai et al., Genes and Dev., 2:1144, 1988.
Sandberg and Larsson, BMC Bioinformatics; 8:48; 2007.
Satake et al., J. Virology, 62:970, 1988.
Schaffner et al., J Mol. Biol., 201:81, 1988.
Schroeder et al., Mol Med 4(2), 72-86, 1998.
Searle et al., Mol. Cell. Biol., 5:1480, 1985.
Sebastian et al., J Immunol (Baltimore, Md.: 1950), 196:144-55, 2016.
Sharp et al., Cell, 59:229, 1989.
Shaul et al., EMBO J., 6:1913, 1987.
Sherman et al., Mol. Cell. Biol., 9:50, 1989.
Shieh and Hung, Stat Appl Genet Mol Biol 8, Article 13. doi: 10.2202/1544-6115.1426, 2009.
Shiina et al., J Hum Genet 54(1), 15-39. doi: 10.1038/jhg.2008.5, 2009.
Shiina et al., Tissue Antigens 64(6), 631-649. doi: 10.1111/j.1399-0039.2004.00327.x, 2004.
Sievers et al., Molecular Systems Biology; 7:539; 2011.
Sleigh et al., J. EMBO, 4:3831, 1985.
Small & Olson, Nature 469(7330):336-342, 2011.
Spalholz et al., Cell, 42:183, 1985.
Spandau et al., J. Virology, 62:427, 1988.
Spandidos & Wilkie, EMBO J., 2:1193, 1983.
Stephens et al., Biochem. 1, 248:1, 1987.
Stewart et al., Genome Res 14(6), 1176-1187. doi: 10.1101/gr.2188104, 2004.
Storey and Tibshirani, Proc. Nat'l Acad. Sci. USA, 100:9440-5, 2003.
Storey, J Royal Statistical Society: Series B (Statistical Methodology), 64:479-98, 2002.
Stuart et al., Nature, 317:828, 1985.
Suarez et al., Circ. Res., 100:1164-1173, 2007.
Sullivan et al., Mol. Cell. Biol., 7:3315, 1987.
Swartzendruber et al., J. Cell. Physiology, 85:179, 1975.
Takebe et al., Mol. Cell. Biol., 8:466, 1988.
Tavernier et al., Nature, 301:634, 1983.
Taylor et al., J. Biol. Chem., 264:15160, 1989.
Taylor et al., Mol. Cell. Biol., 10:165, 1990a.
Taylor et al., Mol. Cell. Biol., 10:176, 1990b.
Temin, In: Gene Transfer, Kucherlapati (Ed.), NY, Plenum Press, 149-188, 1986.
Thiesen et al., J. Virology, 62:614, 1988.
Traheme, et al., PLoS Genetics; 2:e9; 2006.
Tronche et al., Mol. Biol. Med., 7:173, 1990.
Tronche et al., Mol. Cell Biol., 9(11):4759-4766, 1989.
Trudel et al., Genes and Dev., 6:954, 1987.
Tur-Kaspa et al., Mol. Cell Biol., 6:716-718, 1986.
Tusher et al., Proc. Nat′ Acad. Sci. USA, 98:5116-21, 2001.
Tyndell et al., Nuc. Acids. Res., 9:6231, 1981.
Vannice et al., J. Virology, 62:1305, 1988.
Vasseur et al., Proc Natl. Acad. Sci. USA, 77:1068, 1980.
Vigorito et al., Immunity 27(6), 847-859. doi: 10.1016/j.immuni.2007.10.009, 2007.
Wagner et al., Proc. Natl. Acad. Sci. USA 87(9):3410-3414, 1990.
Wang & Olson, Curr. Opin. Genet. Dev. 19(3):205-211, 2009.
Wang et al., Cell, 47:241, 1986.
Wang et al., Dev Cell 15(2):261-271, 2008b.
Wang et al., Proc. Natl. Acad. Sci. USA, 105:7738-7743, 2008a.
Weber et al., Cell, 36:983, 1984.
Weinberger et al. Mol. Cell. Biol., 8:988, 1984.
Wen et al., PLoS Comput Biol 11(9), e1004441. doi: 10.1371/journal.pcbi.1004441, 2015.
Winoto et al., Cell, 59:649, 1989.
Winter et al., Nat Cell Biol 11(3), 228-234. doi: 10.1038/ncb0309-228, 2009.
Wong et al., Gene, 10:87-94, 1980.
Wu and Wu, Biochemistry, 27: 887-892, 1988.
Wu and Wu, J. Biol. Chem., 262:4429-4432, 1987.
Wu et al., Transfusion, 54:169-78, 2014.
Xu et al., Curr. Biol., 13:790-795, 2003.
Yang et al., Nat. Med. 13:486-491, 2007.
Yang et al., Proc. Natl. Acad. Sci. USA, 87:4144-4148, 1990.
Yel, L. Journal of Clinical Immunology 30(1), 10-16. doi: 10.1007/s10875-009-9357-x, 2010.
Yutzey et al. Mol. Cell. Biol., 9:1397, 1989.
Zelenin et al., FEBS Lett, 287(1-2):118-120, 1991.
Zeng et al., Cancer Res., 62(13):3630-3635, 2002.
Zhou et al., PLoS Pathog 5(12):e1000681, 2009.

1. A method of identifying a subject having or at risk of developing an immune or inflammatory disorder comprising (a) assessing the level of HSA-miR-6891-5p in a sample from said subject, and (b) comparing the level of HSA-miR-6891-5p in said sample with a normal sample or predetermined control level, wherein an altered level of HSA-miR-6891-5p indicates the existence of or increased risk for an immune or inflammatory disorder.

2. The method of claim 1, wherein the HSA-miR-6891-5p level is elevated.

3. The method of claim 1, wherein the HSA-miR-6891-5p level is reduced.

4. The method of claim 1, wherein the sample is a blood sample.

5. The method of claim 1, wherein said inflammatory disorder is cancer.

6. The method of claim 1, wherein said immune disorder is an autoimmune disorder.

7. The method of claim 1, wherein said immune or inflammatory disorder is selected from obesity, Crohn's disease, rheumatoid arthritis, asthma, autoimmune thyroid disease, blastic crisis, alopecia areata, multiple sclerosis, autoimmune hepatitis, Addison's disease, type 1 diabetes, type 2 diabetes, bladder cancer, chronic obstructive pulmonary disease, Grave's disease, systemic lupus erythematosus, lung cancer, or Alzheimer's disease.

8. The method of claim 1, wherein said immune disorder is IgA nephropathy or IgA deficiency.

9. The method of claim 1, wherein said subject is a non-human animal or a human.

10. (canceled)

11. A method of treating a subject having or at risk of developing an immune or inflammatory disorder comprising administering to said subject an agonist or antagonist of HSA-miR-6891-5p.

12. The method of claim 11, further comprising (a) assessing the level of HSA-miR-6891-5p in a sample from said subject, and (b) comparing the level of HSA-miR-6891-5p in said sample with a normal sample or predetermined control level.

13. The method of claim 11, wherein the HSA-miR-6891-5p level is elevated, and an antagonist is administered.

14. The method of claim 11, wherein the HSA-miR-6891-5p level is reduced, and an agonist is administered.

15. The method of claim 11, wherein said inflammatory disorder is cancer.

16. The method of claim 11, wherein said immune disorder is an autoimmune disorder.

17. The method of claim 11, wherein said immune or inflammatory disorder is selected from obesity, Crohn's disease, rheumatoid arthritis, asthma, autoimmune thyroid disease, blastic crisis, alopecia areata, multiple sclerosis, autoimmune hepatitis, Addison's disease, type 1 diabetes, type 2 diabetes, bladder cancer, chronic obstructive pulmonary disease, Grave's disease, systemic lupus erythematosus, lung cancer, or Alzheimer's disease.

18. The method of claim 11, wherein said immune disorder is IgA nephropathy or IgA deficiency.

19. The method of claim 11, wherein said subject is a non-human animal or a human.

20. (canceled)

21. The method of claim 13, wherein said antagonist is a miR antagomir or antisense molecule.

22. The method of claim 14, wherein said agonist is HSA-miR-6891-5p or a mimic thereof.

23. The method of claim 11, wherein said agonists/antagonist is formulated in a lipid delivery vehicle.

24. The methods of claim 11, wherein said antagonist is a nucleic acid containing at least one non-natural base.

25. The method of claim 1, wherein said agonist/antagonist is administered multiple times.

26. The method of claim 25, wherein said agonist/antagonist is administered daily, every other day, every third day, every fourth day, every fifth day, every sixth day, weekly or monthly or continuously over a time period exceeding 24 hours.

27. (canceled)


SEQUENCE LISTING (20 sequences)

SEQ ID NO: 1 - RNA, 93 nt, Artificial Sequence
  Synthetic Primer
guaaggagggggaugaggggucauaucucuucucagggaaagcaggagcccuucagcagggucagggccccucaucuuccccuccuuucccag

SEQ ID NO: 2 - RNA, 19 nt, Artificial Sequence
  Synthetic primer
uaaggagggggaugagggg

SEQ ID NO: 3 - RNA, 21 nt, Artificial Sequence
  Synthetic primer
cccucaucuuccccuccuuuc

SEQ ID NO: 4 - DNA, 93 bp, Artificial Sequence
  Synthetic primer
gtaaggagggggatgaggggtcatatctcttctcagggaaagcaggagcccttcagcagggtcagggcccctcatcttcccctcctttcccag

SEQ ID NO: 5 - DNA, 104 bp, Artificial Sequence
  Synthetic primer
gtaaggagggggatgaggggtcatatctcttctcagggaaagcaggagcccttctggagcccttcagcagggtcagggcccctcgtcttcccctcctttcccag

SEQ ID NO: 6 - DNA, 93 bp, Artificial Sequence
  Synthetic primer
gtaaggagggggatgaggggtcatatctcttctcagggaaagcaggagcccttcagcagggtcagggcccctcatcttcccttcctttcccag

SEQ ID NO: 7 - DNA, 105 bp, Artificial Sequence
  Synthetic primer
gtaaggagggggatgaggggtcatatctgttctcagggaaagcaggagcccttctggagccccttcagcagggtcagggcccctcatcttcccctcctttcccag

SEQ ID NO: 8 - DNA, 104 bp, Artificial Sequence
  Synthetic primer
gtaaggagggggatgaggggtcatatctcttctcagggaaagcaggagcccttctggagcccttcagcagggtcagggcccctcatcttcccctcctttcccag

SEQ ID NO: 9 - DNA, 104 bp, Artificial Sequence
  Synthetic primer
gtaaggagggggatgaggggtcatatctcttctcagggaaagcaggagcccttctggagcccttcagcagggtcaggacccctcatcttcccctcctttcccag

SEQ ID NO: 10 - DNA, 93 bp, Artificial Sequence
  Synthetic primer
gtaaggagggggatgaggggtcatatctcttctcagggaaagcaggagcccttcagcagggtcagggcccctcatcttcccttcctttcccgg

SEQ ID NO: 11 - DNA, 104 bp, Artificial Sequence
  Synthetic primer
gtaaggagggggatgaggggtcatatctcttctcagggaaagcaggagcccttctggagcccttcagcagggtcggggcccctcatcttcccctcctttcccag

SEQ ID NO: 12 - RNA, 48 nt, Artificial Sequence
  Synthetic primer
gccgcccgccuguccccaccccugaauaaacuccaugcucccccaagc

SEQ ID NO: 13 - RNA, 19 nt, Artificial Sequence
  Synthetic primer
ggggaguagggggaggaau

SEQ ID NO: 14 - RNA, 54 nt, Artificial Sequence
  Synthetic primer
cucgaggccgcccgccucuccccaauauugcauaaacuccaugcucccccaagc

SEQ ID NO: 15 - DNA, 20 bp, Artificial Sequence
  Synthetic primer
cagaaacttcccacctagcc

SEQ ID NO: 16 - DNA, 20 bp, Artificial Sequence
  Synthetic primer
ctgggattcgtgtagtgctt

SEQ ID NO: 17 - DNA, 20 bp, Artificial Sequence
  Synthetic primer
agagctacgagctgcctgac

SEQ ID NO: 18 - DNA, 20 bp, Artificial Sequence
  Synthetic primer
agcactgtgttggcgtacag

SEQ ID NO: 19 - DNA, 17 bp, Artificial Sequence
  Synthetic primer
cagaccgccaagctgaa

SEQ ID NO: 20 - DNA, 20 bp, Artificial Sequence
  Synthetic primer
gagccgtacatgaactgagg